SEQUENCE LISTING (3 sequences)

SEQ ID NO: 1 - protein, 1279 aa, Artificial Sequence
  Description of Artificial Sequence Synthetic polypeptide
MDWTWILFLVAAATRVHSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3837 bp, Artificial Sequence
  Description of Artificial Sequence Synthetic polynucleotide
atggattggacttggattctctttctcgttgctgcagccacacgcgttcatagcagccagtgtgtgaacctgaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatccacgtgtctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgccctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtggaaggcttcaactgctacttccctctgcagagctacggcttccagccaacaaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaagatgtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagcccaagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggacaaggtggaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacaca

SEQ ID NO: 3 - DNA, 6797 bp, Artificial Sequence
  Description of Artificial Sequence Synthetic polynucleotide
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacttggattctctttctcgttgctgcagccacacgcgttcatagcagccagtgtgtgaacctgaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatccacgtgtctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgccctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtggaaggcttcaactgctacttccctctgcagagctacggcttccagccaacaaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaagatgtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagcccaagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggacaaggtggaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

(19) United States
(12) Patent Application Publication
(10) Pub. No.: US 2022/0331421 A1
(43) Pub. Date: Oct. 20, 2022

(54) Methods of Inducing Immune Response Against Severe Acute Respiratory Syndrome Coronavirus 2 (SARS-CoV-2) Variants of Concern

(71) Applicant: Inovio Pharmaceuticals, Inc., Plymouth Meeting, PA (US)

(72) Inventors: Trevor Smith, San Diego, CA (US); Stephanie Ramos, Santee, CA (US); Viviane Machado De Mello Andrade, San Diego, CA (US)

(21) Appl. No.: 17/720,025

(22) Filed: Apr. 13, 2022

Related U.S. Application Data
(60) Provisional application No. 63/174,375, filed on Apr. 13, 2021.
(60) Provisional application No. 63/215,172, filed on Jun. 25, 2021.
(60) Provisional application No. 63/247,707, filed on Sep. 23, 2021.
(60) Provisional application No. 63/309,387, filed on Feb. 11, 2022.
(60) Provisional application No. 63/314,074, filed on Feb. 25, 2022.

(51) Int. Cl. A61K 39/215; A61P 31/14; C12N 15/86

(52) U.S. Cl. CPC A61K 39/215; A61P 31/14; C12N 15/86; A61K 2039/53

(57) ABSTRACT

Disclosed herein are methods of administering and uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof to induce an immune response against Severe Acute Respiratory Syndrome coronavirus 2 (SARS-CoV-2) variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529.

### CROSS REFERENCE TO RELATED APPLICATIONS

This application claims the benefit of U.S. provisional application No. 63/174,375, filed Apr. 13, 2021; U.S. provisional application No. 63/215,172, filed Jun. 25, 2021; U.S. provisional application No. 63/247,707, filed Sep. 23, 2021; U.S. provisional application No. 63/309,387, filed Feb. 11, 2022; and U.S. provisional application No. 63/314,074 filed Feb. 25, 2022. Each of these applications is incorporated by reference herein in its entirety.

### SEQUENCE LISTING

The instant application contains a Sequence Listing which has been submitted electronically in ASCII format and is hereby incorporated by reference in its entirety. Said ASCII copy, created on Apr. 13, 2022, is named 104409_000681_SL.txt and is 25,862 bytes in size.

### TECHNICAL FIELD

The present invention relates to methods of administering a plasmid encoding residues 19-1279 of SEQ ID NO: 1, a plasmid comprising nucleotides 55-3837 of SEQ ID NO: 2, pGX9501, INO-4800 drug product, or a biosimilar thereof to induce an immune response against Severe Acute Respiratory Syndrome coronavirus 2 (SARS-CoV-2) variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529.

### BACKGROUND

SARS-CoV-2, the causative agent of the COVID-19 pandemic continues to cause unprecedented levels of mortality and socioeconomic burden. Concerningly, virus surveillance shows the global spread of novel SARS-CoV-2 variants, which are more infectious and displaying increased transmissibility and pathology [Chen, R. E., et al., Resistance of SARS-CoV-2 variants to neutralization by monoclonal and serum-derived polyclonal antibodies. Nat Med, 2021; Davies, N. G., et al., Increased mortality in community-tested cases of SARS-CoV-2 lineage B.1.1.7. Nature, 2021; Davies, N. G., et al., Estimated transmissibility and impact of SARS-CoV-2 lineage B.1.1.7 in England. Science, 2021. 372(6538).]. Some of these variants of concern (VOC) contain mutations in the Spike protein receptor binding domain (RBD), the region which interacts with the host ACE2 receptor, and to which many SARS-CoV-2 neutralizing antibodies target. The B.1.1.7 lineage, first emerging VOC reported in the United Kingdom, contains the N501Y and D614G mutations and the del69-70 in the RBD and/or S1 regions, has demonstrated increased transmissibility and pathology, but does not appear to significantly evade neutralizing antibody responses generated by current vaccines approved for use [Wu, K., et al., mRNA-1273 vaccine induces neutralizing antibodies against spike mutants from global SARS-CoV-2 variants. bioRxiv, 2021; Xie, X., et al., Neutralization of SARS-CoV-2 spike 69/70 deletion, E484K and N501Y variants by BNT162b2 vaccine-elicited sera. Nat Med, 2021.]. The B.1.351 (South Africa variant) and P.1 (Brazil variant) lineages have additional mutations, including E484K in the RBD region [Wibmer, C. K., et al., SARS-CoV-2 501Y.V2 escapes neutralization by South African COVID-19 donor plasma. bioRxiv, 2021; Wang, Z., et al., mRNA vaccine-elicited antibodies to SARS-CoV-2 and circulating variants. Nature, 2021; Garcia-Beltran, W. F., et al., Multiple SARS-CoV-2 variants escape neutralization by vaccine-induced humoral immunity. Cell, 2021.]. Several VOCs have emerged in India that have been associated with increased transmissibility and resistance to neutralization, including the double mutant B.1.617.1 and the delta variant B.1.617.2.

Notably, sera isolated from convalescent individuals and vaccinees exposed to the wild-type (WT) Spike protein sequence (GenBank RefSeq sequence NC_045512.2 from Wuhan (China)) have shown significantly lower levels of neutralizing activity against the B.1.351 and P.1 variants [Garcia-Beltran, W. F., et al., Multiple SARS-CoV-2 variants escape neutralization by vaccine-induced humoral immunity. Cell, 2021; Wang, P., et al., Increased Resistance of SARS-CoV-2 Variants B.1.351 and B.1.1.7 to Antibody Neutralization. bioRxiv, 2021; Edara, V. V., et al., Reduced binding and neutralization of infection- and vaccine-induced antibodies to the B.1.351 (South African) SARS-CoV-2 variant. bioRxiv, 2021; Madhi, S. A., et al., Efficacy of the ChAdOx1 nCoV-19 Covid-19 Vaccine against the B.1.351 Variant. N Engl J Med, 2021; Mahase, E., Covid-19: Novavax vaccine efficacy is 86% against UK variant and 60% against South African variant. 2021. 372: p. n296.].

### SUMMARY

Provided herein are methods of inducing an immune response against Severe Acute Respiratory Syndrome coronavirus 2 (SARS-CoV-2) variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 in a subject in need thereof by administering to the subject an effective amount of a plasmid encoding residues 19-1279 of SEQ ID NO: 1, a plasmid comprising nucleotides 55-3837 of SEQ ID NO: 2, pGX9501, INO-4800 drug product, or a biosimilar thereof. Also provided herein are methods of protecting a subject in need thereof from infection with SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529, the method comprising administering to the subject an effective amount of a plasmid encoding residues 19-1279 of SEQ ID NO: 1, a plasmid comprising nucleotides 55-3837 of SEQ ID NO: 2, pGX9501, INO-4800 drug product, or a biosimilar thereof. Further provided are methods of treating an infection with SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 in a subject in need thereof, the method comprising administering to the subject an effective amount of a plasmid encoding residues 19-1279 of SEQ ID NO: 1, a plasmid comprising nucleotides 55-3837 of SEQ ID NO: 2, pGX9501, INO-4800 drug product, or a biosimilar thereof. Also provided herein are methods for treating or protecting a subject against a disease or disorder associated with infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 by administering to the subject an effective amount of a plasmid encoding residues 19-1279 of SEQ ID NO: 1, a plasmid comprising nucleotides 55-3837 of SEQ ID NO: 2, pGX9501, INO-4800 drug product, or a biosimilar thereof. In some embodiments, the disease or disorder associated with infection by a SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 is Coronavirus Disease 2019 (COVID-19), Multisystem inflammatory syndrome in adults (MIS-A), or Multisystem inflammatory syndrome in children (MIS-C). In any of these methods, the administering may include at least one of electroporation and injection. According to some embodiments, the administering comprises parenteral administration, for example by intradermal, intramuscular, or subcutaneous injection, optionally followed by electroporation. In some embodiments of the disclosed methods, an initial dose of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof is administered to the subject, optionally the initial dose is 0.5 mg, 1.0 mg or 2.0 mg of the plasmid encoding residues 19-1279 of SEQ ID NO: 1, plasmid comprising nucleotides 55-3837 of SEQ ID NO: 2, pGX9501, INO-4800 drug product, or a biosimilar thereof. The methods may further involve administration of a subsequent dose of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof to the subject about four weeks after the initial dose, optionally wherein the subsequent dose is 0.5 mg, 1.0 mg or 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof. In still further embodiments, the methods involve administration of one or more further subsequent doses of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof to the subject at least twelve weeks after the initial dose, optionally wherein the further subsequent dose is 0.5 mg, 1.0 mg, or 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof.

Provided herein are uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof in a method of inducing an immune response against SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 in a subject in need thereof. Also provided herein are uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof in a method of protecting a subject in need thereof from infection with SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. Further provided are uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof in a method of treating an infection with SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 in a subject in need thereof. Also provided herein are uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof in a method of treating or protecting a subject against a disease or disorder associated with infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. In some embodiments, the disease or disorder associated with infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 is Coronavirus Disease 2019 (COVID-19), Multisystem inflammatory syndrome in adults (MIS-A), or Multisystem inflammatory syndrome in children (MIS-C). In accordance with any of these uses, a plasmid encoding residues 19-1279 of SEQ ID NO: 1, a plasmid comprising nucleotides 55-3837 of SEQ ID NO: 2, pGX9501, INO-4800 drug product, or a biosimilar thereof may be administered to the subject by at least one of electroporation and injection. In some embodiments, a plasmid encoding residues 19-1279 of SEQ ID NO: 1, a plasmid comprising nucleotides 55-3837 of SEQ ID NO: 2, pGX9501, INO-4800 drug product, or a biosimilar thereof is parenterally administered to the subject, for example by intradermal, intramuscular, or subcutaneous injection, optionally followed by electroporation. In some embodiments of the disclosed uses, an initial dose of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof is administered to the subject, optionally the initial dose is 0.5 mg, 1.0 mg or 2.0 mg of INO-4800 or a biosimilar thereof. The uses may further involve administration of a subsequent dose of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof to the subject about four weeks after the initial dose, optionally wherein the subsequent dose is 0.5 mg,1.0 mg or 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof. In still further embodiments, the uses involve administration of one or more further subsequent doses of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof to the subject at least twelve weeks after the initial dose, optionally wherein the further subsequent dose is 0.5 mg, 1.0 mg, or 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof.

Further provided herein are uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof in the preparation of a medicament for treating or protecting against infection with SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. In some embodiments, the medicament is for treating or protecting against a disease or disorder associated with infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. In some embodiments, the disease or disorder associated with infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 is Coronavirus Disease 2019 (COVID-19), Multisystem inflammatory syndrome in adults (MIS-A), or Multisystem inflammatory syndrome in children (MIS-C).

### BRIEF DESCRIPTION OF THE DRAWINGS

FIGS. 1A and 1B show humoral antibody cross-reactivity responses against SARS-CoV-2 variants. In FIG. 1A, sera from Phase 1 INO-4800 vaccinees were assessed by ELISA for IgG binding to WT (Wuhan), B.1.1.7, B.1.351, and P.1 variant Spike protein (S1 and S2). Data points indicate endpoint titers for an individual study sample variants (n=4, 0.5 mg vaccine dose; n=5, 1.0 mg; n=11, 2.0 mg) and were calculated as the titer that exhibited an OD 3 SD above baseline. FIG. 1B shows SARS-CoV-2 pseudovirus neutralization ID50 titers for sera samples from 13 or 12 Phase 1 INO-4800 vaccinees comparing WT (Wuhan) against B.1.1.7, B.1.351, P.1, and B.1.617.2. Each data point represents the mean of technical duplicates for each individual (n=1, 0.5 mg vaccine dose; n=4, 1.0 mg; n=8, 2.0 mg). Dotted lines indicate the limit of detection of 16. ns—not significant, *P<0.05, ***P<0.0001 (Wilcoxon signed-rank test).

FIG. 2 shows INO-4800 cellular immune response against SARS-CoV-2 variants. PBMCs from 10 Phase 1 subjects were collected 8 weeks after receiving the second dose of INO-4800 (n=5, 1.0 mg; n=5, 2.0 mg). PBMCs were treated with peptide pools spanning the entire Spike proteins of the WT, B.1.1.7, B.1.351, or P.1 variants and cellular responses were measured by IFNγ ELISpot assay. Mean±s.e.m. IFNγ SFUs/million PBMCs of experimental triplicates are shown. ns—not significant (Wilcoxon signed-rank test).

FIGS. 3A-3C show schematic diagrams and molecular modeling of SARS-CoV-2 spike proteins. FIG. 3A provides a Spike protein diagram with major features labeled: N-terminal domain (NTD), receptor binding domain (RBD), fusion peptide (FP), heptad repeats 1 and 2 (HR1 and HR2), transmembrane region (™), C-terminal domain (CT). FIG. 3B provides molecular models of spike protein with mutations indicated for B.1.1.7, B.1.351, and P.1 variants. Trimer model is depicted with one subunit as a Ca trace and colored identically to the diagram in FIG. 3A with the two subunits outlined for clarity. Large loops not modeled are indicated by dashed lines and the stalk and membrane-spanning portion of the molecule are indicated with cylinders. FIG. 3C provides diagrams of spike protein with major features labeled and mutations indicated for B.1.1.7, B.1.351, P.1, and B.1.617.2 variants.

FIG. 4 shows cross-neutralizing antibody responses against SARS-CoV-2 variants. SARS-CoV-2 pseudovirus neutralization ID50 titers for sera samples from 12 Phase 2 INO-4800 vaccinees comparing Wuhan against B.1.617.1. Each data point represents the mean of technical duplicates for each individual. Dotted lines indicate the limit of detection of 16. ***P<0.0001 (Wilcoxon signed-rank test).

FIG. 5 shows INO-4800 cellular immune response against B.1.617.2 delta variant. PBMCs from 10 Phase 1 subjects were collected 8 weeks after receiving the second dose of INO-4800. PBMCs were treated with peptide pools spanning the entire Spike proteins of the Wuhan or B.1.617.2 variants and cellular responses were measured by IFNγ ELISpot assay. Mean±s.e.m. IFNγ SFUs/million PBMCs of experimental triplicates are shown. ns—not significant (Wilcoxon signed-rank test).

FIGS. 6A-6D illustrate the study design and durability of humoral immune responses in rhesus macaques primed with INO-4800. FIG. 6A provides a schematic depicting the prime immunization schedule and sample collection timepoints. Note: The longitudinal collection for the NHPs in the 1 mg dose group ended at Week 35 and for 2 mg dose group at Week 52. FIG. 6B shows longitudinal serum IgG binding titers in rhesus macaques vaccinated with 1 or 2 mg INO-4800 at weeks 0 and 4. Antibody titers in the sera were measured against the wildtype SARS-CoV-2 Spike protein antigen. Antibody titers in the sera were also measured against the SARS-CoV-2 S1, SARS-CoV-2 S2 and RBD proteins (FIG. 6D). FIG. 6C shows longitudinal pseudovirus neutralizing activity (ID₅₀) in NHPs primed with INO-4800, measured against SARS-CoV-2 pseudotyped viral stocks for the ancestral (wild-type; Wuhan-Hu-1) SARS-CoV-2 as well as Alpha (B.1.1.7), Beta (B.1.351), and Gamma (P.1) pseudoviruses.

FIGS. 7A-7D illustrate humoral immune responses following homologous boost in INO-4800-primed rhesus macaques. Antibody responses were measured in animals boosted with 1 mg of INO-4800 on the day of the boost (week 0) and at weeks 2 and 4 post-boost. Solid lines represent geometric mean titers (GMT) or geometric mean inhibition (GMI). FIG. 7A provides a schematic of the boost schedule with the respective animal IDs. FIG. 7B shows serum IgG binding titers measured against the ancestral, Beta, Delta, Gamma, and Omicron Spike proteins. FIG. 7C illustrates serum pseudovirus neutralizing activity measured against the ancestral, Beta, Delta, Gamma, and Omicron pseudoviruses. FIG. 7D shows ACE2 blocking activity in the serum measured against the ancestral, Beta, Delta, and Gamma Spike proteins.

FIGS. 8A-8F illustrate cellular immune responses following homologous boost in INO-4800-primed rhesus macaques. T cell responses were measured in animals boosted with 1 mg of INO-4800 on the day of the boost (week 0) and at week 2 post-boost. FIGS. 8A-8C show CD4 and FIGS. 8D-F show CD8 T cell responses in INO-4800-boosted animals against ancestral or Beta derived peptide pools. The sum of IFNγ, IL-2, and TNF responses are represented in FIGS. 8C and 8F. Bars represent median.

FIGS. 9A-9C show INO-4800 cellular mediated immunity against SARS-CoV-2 Omicron variant. In FIG. 9A, PBMCs from 13 Phase 1 subjects were collected 8 weeks after receiving the second dose of INO-4800 (0.5 mg, n=4; 1.0 mg, n=4; 2.0 mg, n=5). PBMCs were treated with peptide megapools spanning the entire Spike proteins of the ancestral (WT) and Omicron variant, and cellular responses were measured by IFNγ ELISpot assay. Graphs depict individual subject responses as IFNγ SFUs/million PBMCs. In FIG. 9B, PBMCs from 11 Phase 1 subjects, selected from the subset used for the IFNγ ELISpot assay, were evaluated for SARS-CoV-2 spike specific cytokine production by CD4 and CD8 T cells via flow cytometry. Graphs depict the frequency of individual cytokines being produced after stimulation with the WT or Omicron megapools. FIG. 9C illustrates the functional profile of cytokine producing Central Memory (CM), Effector Memory (EM), or Effector (E) T cells are depicted in the pie charts for all evaluable subjects. Statistical analyses were performed on all paired datasets t. ns: not significant; *: P<0.05 (Wilcoxon signed-rank test).

FIGS. 10A and 10B show humoral antibody cross-reactivity responses against SARS-CoV-2 Omicron variant. In FIG. 10A, sera from Phase 1 and 2 INO-4800 vaccinees were assessed by ELISA for IgG binding to WT and Omicron variant full-length Spike (S1 and S2 trimer) and RBD proteins. Data points represent for individual study participants (vaccine dose: 1.0 mg, n=3; 2.0 mg, n=7). In FIG. 10B, SARS-CoV-2 pseudovirus neutralization ID₅₀ titers for sera samples from 12 Phase 1 and Phase 2 INO-4800 vaccinees comparing WT against Omicron. Each data point represents the mean of technical duplicates for each individual (vaccine dose: 0.5 mg, n=1; 1.0 mg, n=3; 2.0 mg, n=8). Dotted lines indicate the limit of detection of 8. ns: not significant, **: P<0.001 (Wilcoxon signed-rank test).

### DETAILED DESCRIPTION OF ILLUSTRATIVE EMBODIMENTS

Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The term “comprising” is intended to include examples encompassed by the terms “consisting essentially of” and “consisting of”; similarly, the term “consisting essentially of” is intended to include examples encompassed by the term “consisting of.” The present disclosure also contemplates other embodiments “comprising,” “consisting of” and “consisting essentially of” the embodiments or elements presented herein, whether explicitly set forth or not.

It is to be appreciated that certain features of the disclosed materials and methods which are, for clarity, described herein in the context of separate embodiments, may also be provided in combination in a single embodiment. Conversely, various features of the disclosed materials and methods that are, for brevity, described in the context of a single embodiment, may also be provided separately or in any subcombination.

The singular forms “a,” “and” and “the” include plural references unless the context clearly dictates otherwise.

The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value. When values are expressed as approximations by use of the antecedent “about,” it will be understood that the particular value forms another embodiment. Reference to a particular numerical value includes at least that particular value unless the context clearly dictates otherwise.

“Adjuvant” as used herein means any molecule added to the vaccine described herein to enhance the immunogenicity of the antigen.

“Antibody” as used herein means an antibody of classes IgG, IgM, IgA, IgD or IgE, or fragments, fragments or derivatives thereof, including Fab, F(ab′) 2, Fd, and single chain antibodies, diabodies, bispecific antibodies, bifunctional antibodies and derivatives thereof. The antibody can be an antibody isolated from the serum sample of mammal, a polyclonal antibody, affinity purified antibody, or mixtures thereof which exhibits sufficient binding specificity to a desired epitope or a sequence derived therefrom.

The term “biosimilar” (of an approved reference product/biological drug, i.e., reference listed drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extent the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Immune response” as used herein means the activation of a host's immune system, e.g., that of a mammal, in response to the introduction of antigen. The immune response can be in the form of a cellular or humoral response, or both.

The INO-4800 drug product (or INO-4800 vaccine) contains 10 mg/mL of the DNA plasmid pGX9501 (SEQ ID NO: 3) in 1×SSC buffer (150 mM sodium chloride and 15 mM sodium citrate).

“Nucleic acid” or “oligonucleotide” or “polynucleotide” or “nucleic acid molecule” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double-stranded or can contain portions of both double-stranded and single-stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, and CMV IE promoter.

“Signal peptide” and “leader sequence” are used interchangeably herein and refer to an amino acid sequence that can be linked at the amino terminus of a SARS-CoV-2 protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that wants or is in need of being immunized with a herein described immunogenic composition or vaccine. The mammal can be a human, chimpanzee, guinea pig, dog, cat, horse, cow, mouse, rabbit, or rat.

“Treatment” or “treating,” as used herein can mean protecting of an animal from a disease through means of preventing, suppressing, repressing, or completely eliminating the disease. Preventing the disease involves administering an immunogenic composition or a vaccine of the present invention to an animal prior to onset of the disease. Suppressing the disease involves administering an immunogenic composition or a vaccine of the present invention to an animal after induction of the disease but before its clinical appearance. Repressing the disease involves administering an immunogenic composition or a vaccine of the present invention to an animal after clinical appearance of the disease.

As used herein, unless otherwise noted, the term “clinically proven” (used independently or to modify the terms “safe” and/or “effective”) shall mean that it has been proven by a clinical trial wherein the clinical trial has met the approval standards of U.S. Food and Drug Administration, EMA or a corresponding national regulatory agency. For example, proof may be provided by the clinical trial(s) described in the examples provided herein.

The term “clinically proven safe”, as it relates to a dose, dosage regimen, treatment or method with a SARS-CoV-2 antigen (for example, a SARS-CoV-2 spike antigen administered as a plasmid encoding residues 19-1279 of SEQ ID NO: 1, a plasmid comprising nucleotides 55-3837 of SEQ ID NO: 2, pGX9501 or INO-4800 or a biosimilar thereof) refers to a favorable risk:benefit ratio with an acceptable frequency and/or acceptable severity of treatment-emergent adverse events (referred to as AEs or TEAEs) compared to the standard of care or to another comparator. An adverse event is an untoward medical occurrence in a patient administered a medicinal product.

The terms “clinically proven efficacy” and “clinically proven effective” as used herein in the context of a dose, dosage regimen, treatment or method refer to the effectiveness of a particular dose, dosage or treatment regimen. Efficacy can be measured based on change in the course of the disease in response to an agent of the present invention. For example, a SARS-CoV-2 antigen (for example, a SARS-CoV-2 spike antigen administered as a plasmid encoding residues 19-1279 of SEQ ID NO: 1, a plasmid comprising nucleotides 55-3837 of SEQ ID NO: 2, pGX9501 or INO-4800 or a biosimilar thereof) is administered to a patient in an amount and for a time sufficient to induce an improvement, preferably a sustained improvement, in at least one indicator that reflects the severity of the disorder that is being treated. Various indicators that reflect the extent of the subject's illness, disease or condition may be assessed for determining whether the amount and time of the treatment is sufficient. Such indicators include, for example, clinically recognized indicators of disease severity, symptoms, or manifestations of the disorder in question. The degree of improvement generally is determined by a physician, who may make this determination based on signs, symptoms, biopsies, or other test results, and who may also employ questionnaires that are administered to the subject, such as quality-of-life questionnaires developed for a given disease. For example, a SARS-CoV-2 antigen (for example, a SARS-CoV-2 spike antigen administered as a plasmid encoding residues 19-1279 of SEQ ID NO: 1, a plasmid comprising nucleotides 55-3837 of SEQ ID NO: 2, pGX9501 or INO-4800 or a biosimilar thereof) may be administered to achieve an improvement in a patient's condition related to a SARS-CoV-2 infection. Improvement may be indicated by an improvement in an index of disease activity, by amelioration of clinical symptoms or by any other measure of disease activity.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Provided herein are methods of treating, protecting against, and/or preventing infection with Severe Acute Respiratory Syndrome coronavirus 2 (SARS-CoV-2) 501Y.V2 (also known as B.1.351; South African; or Beta variant), SARS-CoV-2 20I/501Y.V1 (also known as VOC 202012/01; B.1.1.7; United Kingdom; or Alpha variant), SARS-CoV-2 variant P.1 (also known as Brazilian or Gamma variant), SARS-CoV-2 variant B.1.617.1, SARS-CoV-2 variant B.1.617.2 (also known as Delta variant), or SARS-CoV-2 variant B.1.1.529 (also known as Omicron variant) in a subject in need thereof by administering a plasmid encoding residues 19-1279 of SEQ ID NO: 1, a plasmid comprising nucleotides 55-3837 of SEQ ID NO: 2, pGX9501, INO-4800 drug product, or a biosimilar thereof to the subject. Administration of a plasmid encoding residues 19-1279 of SEQ ID NO: 1, a plasmid comprising nucleotides 55-3837 of SEQ ID NO: 2, pGX9501, INO-4800 drug product, or a biosimilar thereof to the subject can induce or elicit an immune response in the subject. The immune response may be a cellular immune response, a humoral immune response, or both. Also provided herein are methods for treating or protecting a subject against a disease or disorder associated with infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 by administering a plasmid encoding residues 19-1279 of SEQ ID NO: 1, a plasmid comprising nucleotides 55-3837 of SEQ ID NO: 2, pGX9501, INO-4800 drug product, or a biosimilar thereof to the subject. In some embodiments, the disease or disorder associated with infection by a SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 is Coronavirus Disease 2019 (COVID-19), Multisystem inflammatory syndrome in adults (MIS-A), or Multisystem inflammatory syndrome in children (MIS-C).

Further provided are methods of inducing an immune response against SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 in a subject in need thereof by administering to the subject an effective amount of a plasmid encoding residues 19-1279 of SEQ ID NO: 1, a plasmid comprising nucleotides 55-3837 of SEQ ID NO: 2, pGX9501, INO-4800 drug product, or a biosimilar thereof. The immune response may be a cellular immune response, a humoral immune response, or both.

In any of these methods, the administering may include at least one of electroporation and injection. According to some embodiments, the administering comprises parenteral administration, for example by intradermal, intramuscular, or subcutaneous injection, optionally followed by electroporation. In some embodiments of the disclosed methods, an initial dose of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof is administered to the subject, optionally the initial dose is 0.5 mg, 1.0 mg or 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof. The methods may further involve administration of a subsequent dose of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof to the subject about four weeks after the initial dose, optionally wherein the subsequent dose is 0.5 mg,1.0 mg or 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof. In still further embodiments, the methods involve administration of one or more further subsequent doses of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof to the subject at least twelve weeks after the initial dose, optionally wherein the further subsequent dose is 0.5 mg, 1.0 mg, or 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof.

Provided herein are uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof in a method of inducing an immune response against SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 in a subject in need thereof. Also provided herein are uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof in a method of protecting a subject in need thereof from infection with SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. Further provided are uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof in a method of treating an infection with SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 in a subject in need thereof. Also provided herein are uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof in a method of treating or protecting a subject against a disease or disorder associated with infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. In some embodiments, the disease or disorder associated with infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 is Coronavirus Disease 2019 (COVID-19), Multisystem inflammatory syndrome in adults (MIS-A), or Multisystem inflammatory syndrome in children (MIS-C). In accordance with any of these uses, a plasmid encoding residues 19-1279 of SEQ ID NO: 1, a plasmid comprising nucleotides 55-3837 of SEQ ID NO: 2, pGX9501, INO-4800 drug product, or a biosimilar thereof may be administered to the subject by at least one of electroporation and injection. In some embodiments, a plasmid encoding residues 19-1279 of SEQ ID NO: 1, a plasmid comprising nucleotides 55-3837 of SEQ ID NO: 2, pGX9501, INO-4800 drug product, or a biosimilar thereof is parenterally administered to the subject, for example by intradermal, intramuscular, or subcutaneous injection, optionally followed by electroporation. In some embodiments of the disclosed uses, an initial dose of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof is administered to the subject, optionally the initial dose is 0.5 mg, 1.0 mg or 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof. The uses may further involve administration of a subsequent dose of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof to the subject about four weeks after the initial dose, optionally wherein the subsequent dose is 0.5 mg, 1.0 mg or 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof. In still further embodiments, the uses involve administration of one or more further subsequent doses of about 0.5 mg to about 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof to the subject at least twelve weeks after the initial dose, optionally wherein the further subsequent dose is 0.5 mg, 1.0 mg, or 2.0 mg of a plasmid encoding residues 19-1279 of SEQ ID NO: 1, a plasmid comprising nucleotides 55-3837 of SEQ ID NO: 2, pGX9501, INO-4800 drug product, or a biosimilar thereof.

Further provided herein are uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof in the preparation of a medicament for treating or protecting against infection with SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. In some embodiments are provided uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof in the preparation of a medicament for treating or protecting against a disease or disorder associated with infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. In some embodiments, the disease or disorder associated with infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 is Coronavirus Disease 2019 (COVID-19), Multisystem inflammatory syndrome in adults (MIS-A), or Multisystem inflammatory syndrome in children (MIS-C).

In accordance with the methods and uses described herein, the induced immune response can include an induced humoral immune response, an induced cellular immune response, or both. The humoral immune response can be induced by about 1.5-fold to about 16-fold, about 2-fold to about 12-fold, or about 3-fold to about 10-fold. The induced humoral immune response can include IgG antibodies and/or neutralizing antibodies that are reactive to the antigen. The induced cellular immune response can include a CD8+ T cell response, which is induced by about 2-fold to about 30-fold, about 3-fold to about 25-fold, or about 4-fold to about 20-fold.

The disclosed methods and uses of a plasmid encoding residues 19-1279 of SEQ ID NO: 1, a plasmid comprising nucleotides 55-3837 of SEQ ID NO: 2, pGX9501, INO-4800 drug product, or a biosimilar thereof can elicit both humoral and cellular immune responses that target the SARS-CoV-2 antigen in the recipient subject (a subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof). For example, a plasmid encoding residues 19-1279 of SEQ ID NO: 1, a plasmid comprising nucleotides 55-3837 of SEQ ID NO: 2, pGX9501, INO-4800 drug product, or a biosimilar thereof can elicit neutralizing antibodies and immunoglobulin G (IgG) antibodies that are reactive with the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. A plasmid encoding residues 19-1279 of SEQ ID NO: 1, a plasmid comprising nucleotides 55-3837 of SEQ ID NO: 2, pGX9501, INO-4800 drug product, or a biosimilar thereof can also elicit CD8+ and CD4+ T cell responses that are reactive to the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 and produce interferon-gamma (IFN-γ), tumor necrosis factor alpha (TNF-α), interleukin-2 (IL-2), or any combination thereof.

A plasmid encoding residues 19-1279 of SEQ ID NO: 1, a plasmid comprising nucleotides 55-3837 of SEQ ID NO: 2, pGX9501, INO-4800 drug product, or a biosimilar thereof can induce a humoral immune response in the recipient subject. The induced humoral immune response can be specific for the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. The induced humoral immune response can be reactive with the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. The humoral immune response can be induced in the recipient subject by about 1.5-fold to about 16-fold, about 2-fold to about 12-fold, or about 3-fold to about 10-fold. The humoral immune response can be induced in the recipient subject by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

The humoral immune response induced by a plasmid encoding residues 19-1279 of SEQ ID NO: 1, a plasmid comprising nucleotides 55-3837 of SEQ ID NO: 2, pGX9501, INO-4800 drug product, or a biosimilar thereof can include an increased level of neutralizing antibodies associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof as compared to a non-recipient subject (a subject not administered a plasmid encoding residues 19-1279 of SEQ ID NO: 1, a plasmid comprising nucleotides 55-3837 of SEQ ID NO: 2, pGX9501, INO-4800 drug product, or a biosimilar thereof). The neutralizing antibodies can be specific for specific for the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. The neutralizing antibodies can be reactive with the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. The neutralizing antibodies can provide protection against and/or treatment of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 and associated pathologies in the recipient subject.

The humoral immune response induced by a plasmid encoding residues 19-1279 of SEQ ID NO: 1, a plasmid comprising nucleotides 55-3837 of SEQ ID NO: 2, pGX9501, INO-4800 drug product, or a biosimilar thereof can include an increased level of IgG antibodies associated with the recipient subject as compared to a non-recipient subject. These IgG antibodies can be specific for the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. These IgG antibodies can be reactive with the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. The level of IgG antibody associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof can be increased by about 1.5-fold to about 16-fold, about 2-fold to about 12-fold, or about 3-fold to about 10-fold as compared to the subject not administered a plasmid encoding residues 19-1279 of SEQ ID NO: 1, a plasmid comprising nucleotides 55-3837 of SEQ ID NO: 2, pGX9501, INO-4800 drug product, or a biosimilar thereof. The level of IgG antibody associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to the subject not administered a plasmid encoding residues 19-1279 of SEQ ID NO: 1, a plasmid comprising nucleotides 55-3837 of SEQ ID NO: 2, pGX9501, INO-4800 drug product, or a biosimilar thereof.

A plasmid encoding residues 19-1279 of SEQ ID NO: 1, a plasmid comprising nucleotides 55-3837 of SEQ ID NO: 2, pGX9501, INO-4800 drug product, or a biosimilar thereof can induce a cellular immune response in the recipient subject. The induced cellular immune response can be specific for the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. The induced cellular immune response can be reactive to the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. The induced cellular immune response can include eliciting a CD8+ T cell response. The elicited CD8+ T cell response can be reactive with the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. The elicited CD8+ T cell response can be polyfunctional. The induced cellular immune response can include eliciting a CD8+ T cell response, in which the CD8+ T cells produce interferon-gamma (IFN-γ), tumor necrosis factor alpha (TNF-α), interleukin-2 (IL-2), or any combination thereof.

The induced cellular immune response can include an increased CD8+ T cell response associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof as compared to the non-recipient subject. The CD8+ T cell response associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof can be increased by about 2-fold to about 30-fold, about 3-fold to about 25-fold, or about 4-fold to about 20-fold as compared to the subject not administered a plasmid encoding residues 19-1279 of SEQ ID NO: 1, a plasmid comprising nucleotides 55-3837 of SEQ ID NO: 2, pGX9501, INO-4800 drug product, or a biosimilar thereof. The CD8+ T cell response associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the subject not administered a plasmid encoding residues 19-1279 of SEQ ID NO: 1, a plasmid comprising nucleotides 55-3837 of SEQ ID NO: 2, pGX9501, INO-4800 drug product, or a biosimilar thereof.

The induced cellular immune response can include an increased frequency of CD3+CD8+ T cells that produce IFN-γ. The frequency of CD3+CD8+IFN-γ+ T cells associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered a plasmid encoding residues 19-1279 of SEQ ID NO: 1, a plasmid comprising nucleotides 55-3837 of SEQ ID NO: 2, pGX9501, INO-4800 drug product, or a biosimilar thereof.

The induced cellular immune response can include an increased frequency of CD3+CD8+ T cells that produce TNF-α. The frequency of CD3+CD8+TNF-α+ T cells associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to the subject not administered a plasmid encoding residues 19-1279 of SEQ ID NO: 1, a plasmid comprising nucleotides 55-3837 of SEQ ID NO: 2, pGX9501, INO-4800 drug product, or a biosimilar thereof.

The induced cellular immune response can include an increased frequency of CD3+CD8+ T cells that produce IL-2. The frequency of CD3+CD8+IL-2+ T cells associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof can be increased by at least about 0.5-fold, 1.0-fold, 1.5-fold, 2.0-fold, 2.5-fold, 3.0-fold, 3.5-fold, 4.0-fold, 4.5-fold, or 5.0-fold as compared to the subject not administered a plasmid encoding residues 19-1279 of SEQ ID NO: 1, a plasmid comprising nucleotides 55-3837 of SEQ ID NO: 2, pGX9501, INO-4800 drug product, or a biosimilar thereof.

The induced cellular immune response can include an increased frequency of CD3+CD8+ T cells that produce both IFN-γ and tumor necrosis factor alpha (TNF-α). The frequency of CD3+CD8+IFN-γ+TNF-α+ T cells associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof can be increased by at least about 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 160-fold, 170-fold, or 180-fold as compared to the subject not administered a plasmid encoding residues 19-1279 of SEQ ID NO: 1, a plasmid comprising nucleotides 55-3837 of SEQ ID NO: 2, pGX9501, INO-4800 drug product, or a biosimilar thereof.

The cellular immune response induced by the immunogenic composition can include eliciting a CD4+ T cell response. The elicited CD4+ T cell response can be reactive with the spike antigen of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529. The elicited CD4+ T cell response can be polyfunctional. The induced cellular immune response can include eliciting a CD4+ T cell response, in which the CD4+ T cells produce IFN-γ, TNF-α, IL-2, or any combination thereof.

The induced cellular immune response can include an increased frequency of CD3+CD4+ T cells that produce IFN-γ. The frequency of CD3+CD4+IFN-γ+ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the immunogenic composition.

The induced cellular immune response can include an increased frequency of CD3+CD4+ T cells that produce TNF-α. The frequency of CD3+CD4+TNF-α+ T cells associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to the subject not administered a plasmid encoding residues 19-1279 of SEQ ID NO: 1, a plasmid comprising nucleotides 55-3837 of SEQ ID NO: 2, pGX9501, INO-4800 drug product, or a biosimilar thereof.

The induced cellular immune response can include an increased frequency of CD3+CD4+ T cells that produce IL-2. The frequency of CD3+CD4+IL-2+ T cells associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 45-fold, 50-fold, 55-fold, or 60-fold as compared to the subject not administered a plasmid encoding residues 19-1279 of SEQ ID NO: 1, a plasmid comprising nucleotides 55-3837 of SEQ ID NO: 2, pGX9501, INO-4800 drug product, or a biosimilar thereof.

The induced cellular immune response can include an increased frequency of CD3+CD4+ T cells that produce both IFN-γ and TNF-α. The frequency of CD3+CD4+IFN-γ+TNF-α+ associated with the subject administered a plasmid encoding residues 19-1279 of SEQ ID NO: 1, a plasmid comprising nucleotides 55-3837 of SEQ ID NO: 2, pGX9501, INO-4800 drug product, or a biosimilar thereof can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to the subject not administered pGX9501, INO-4800 drug product, or a biosimilar thereof.

The dose of a plasmid encoding residues 19-1279 of SEQ ID NO: 1, a plasmid comprising nucleotides 55-3837 of SEQ ID NO: 2, pGX9501, INO-4800 drug product, or a biosimilar thereof administered in accordance with the methods and uses provided herein can be between 1 μg to 10 mg active component/kg body weight/time, and can be 20 μg to 10 mg component/kg body weight/time. Administration can be every 21, 22, 23, 24, 25, 26, 27, 28, 29, 30, 31, 32, 33, 34, 35, 36, 37, 38, 39, 40 or more days or every 3, 4, 5, 6, 7, 8, 9, 10, 11, 12, 13, 14, 15, 16, 17, 18, 19, 20 or more weeks. The number of doses for effective treatment can be 1, 2, 3, 4, 5, 6, 7, 8, 9, 10, or more.

In accordance with the methods and uses disclosed herein, a plasmid encoding residues 19-1279 of SEQ ID NO: 1, a plasmid comprising nucleotides 55-3837 of SEQ ID NO: 2, pGX9501, INO-4800 drug product, or a biosimilar thereof may be administered, for example, in one, two, three, four, or more injections. In some embodiments, an initial dose of about 0.5 mg to about 2.0 mg of nucleic acid molecule is administered to the subject. The initial dose may be administered in one, two, three, or more injections. The initial dose may be followed by administration of one, two, three, four, or more subsequent doses of about 0.5 mg to about 2.0 mg of the nucleic acid molecule about one, two, three, four, five, six, seven, eight, ten, twelve or more weeks after the immediately prior dose. Each subsequent dose may be administered in one, two, three, or more injections. In some embodiments, a plasmid encoding residues 19-1279 of SEQ ID NO: 1, a plasmid comprising nucleotides 55-3837 of SEQ ID NO: 2, pGX9501, INO-4800 drug product, or a biosimilar thereof is administered to the subject before, with, or after an additional agent. In some embodiments, a plasmid encoding residues 19-1279 of SEQ ID NO: 1, a plasmid comprising nucleotides 55-3837 of SEQ ID NO: 2, pGX9501, INO-4800 drug product, or a biosimilar thereof is administered as a booster following administration of a different agent for the treatment of the SARS-CoV-2 infection or the treatment or prevention of a disease or disorder associated with SARS-CoV-2 infection.

The subject can be a mammal, such as a human, a horse, a nonhuman primate, a cow, a pig, a sheep, a cat, a dog, a guinea pig, a rabbit, a rat, or a mouse.

In accordance with the methods and uses provided herein, a plasmid encoding residues 19-1279 of SEQ ID NO: 1, a plasmid comprising nucleotides 55-3837 of SEQ ID NO: 2, or pGX9501 can be administered as an immunogenic composition further comprising a pharmaceutically acceptable excipient. The pharmaceutically acceptable excipient can be a vehicle, carrier, buffer, or diluent. As used herein. “buffer” refers to a buffered solution that resists changes in pH by the action of its acid-base conjugate components. The buffer generally has a pH from about 4.0 to about 8.0, for example from about 5.0 to about 7.0. In some embodiments, the buffer is saline-sodium citrate (SSC) buffer. In some embodiments, the immunogenic composition comprises 10 mg/mL of a plasmid encoding residues 19-1279 of SEQ ID NO: 1, a plasmid comprising nucleotides 55-3837 of SEQ ID NO: 2, or the DNA plasmid pGX9501 in buffer, preferably SSC buffer.

A plasmid encoding residues 19-1279 of SEQ ID NO: 1, a plasmid comprising nucleotides 55-3837 of SEQ ID NO: 2, pGX9501, INO-4800 drug product, or a biosimilar thereof can be delivered via a variety of routes. Typical delivery routes include parenteral administration, e.g., intradermal, intramuscular or subcutaneous delivery, optionally followed by electroporation as described herein.

Electroporation may be performed such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the vaccine described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the vaccine into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system are described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA® (Inovio Pharmaceuticals, Blue Bell Pa.) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra® device and system is described in U.S. Pat. No. 7,245,963, the contents of which are herein incorporated by reference. The CELLECTRA® device may be the CELLECTRA 2000® device or CELLECTRA® 3PSP device. CELLECTRA® 2000 is configured by the manufacturer to support either ID (intradermal) or IM (intramuscular) administration. The CELLECTRA® 2000 includes the CELLECTRA® Pulse Generator, the appropriate applicator, disposable sterile array and disposable sheath (ID only). The DNA plasmid is delivered separately via needle and syringe injection in the area delineated by the electrodes immediately prior to the electroporation treatment.

The MID may be an Elgen 1000 system (Inovio Pharmaceuticals). The Elgen 1000 system may comprise a device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during electroporation. This is particularly advantageous as it means that the electric field is applied to the same area as the injected fluid. There has traditionally been a problem with electroporation in that it is very difficult to accurately align an electrode with previously injected fluid and so users have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

Use in Combination

In some embodiments, the present invention provides a method of treating SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 infection, or treating, protecting against, and/or preventing a disease or disorder associated with such a SARS-CoV-2 infection in a subject in need thereof by administering a plasmid encoding residues 19-1279 of SEQ ID NO: 1, a plasmid comprising nucleotides 55-3837 of SEQ ID NO: 2, pGX9501, INO-4800 drug product, or a biosimilar thereof in combination with one or more additional agents for the treatment of the SARS-CoV-2 infection or the treatment or prevention of disease or disorder associated with the SARS-CoV-2 infection. In some embodiments, the disease or disorder associated with the SARS-CoV-2 infection is Coronavirus Disease 2019 (COVID-19), Multisystem inflammatory syndrome in adults (MIS-A), or Multisystem inflammatory syndrome in children (MIS-C).

A plasmid encoding residues 19-1279 of SEQ ID NO: 1, a plasmid comprising nucleotides 55-3837 of SEQ ID NO: 2, pGX9501, INO-4800 drug product, or a biosimilar thereof and the additional agent may be administered using any suitable method such that a combination of a plasmid encoding residues 19-1279 of SEQ ID NO: 1, a plasmid comprising nucleotides 55-3837 of SEQ ID NO: 2, pGX9501, INO-4800 drug product, or a biosimilar thereof and the additional agent are both present in the subject. In one embodiment, the method may comprise administration of a first composition comprising an agent for the treatment of SARS-CoV-2 infection or the treatment or prevention of disease or disorder associated with SARS-CoV-2 infection and administration of a plasmid encoding residues 19-1279 of SEQ ID NO: 1, a plasmid comprising nucleotides 55-3837 of SEQ ID NO: 2, pGX9501, INO-4800 drug product, or a biosimilar thereof less than 1, less than 2, less than 3, less than 4, less than 5, less than 6, less than 7, less than 8, less than 9 or less than 10 days following administration of the first composition comprising the agent for the treatment of SARS-CoV-2 infection or the treatment or prevention of disease or disorder associated with SARS-CoV-2 infection. In one embodiment, the method may comprise administration of a plasmid encoding residues 19-1279 of SEQ ID NO: 1, a plasmid comprising nucleotides 55-3837 of SEQ ID NO: 2, pGX9501, INO-4800 drug product, or a biosimilar thereof and administration of a second composition comprising an agent for the treatment of SARS-CoV-2 infection or the treatment or prevention of disease or disorder associated with SARS-CoV-2 infection less than 1, less than 2, less than 3, less than 4, less than 5, less than 6, less than 7, less than 8, less than 9 or less than 10 days following administration of a plasmid encoding residues 19-1279 of SEQ ID NO: 1, a plasmid comprising nucleotides 55-3837 of SEQ ID NO: 2, pGX9501, INO-4800 drug product, or a biosimilar thereof. In one embodiment, the method may comprise concurrent administration of a first composition comprising an agent for the treatment of SARS-CoV-2 infection or the treatment or prevention of disease or disorder associated with SARS-CoV-2 infection and a plasmid encoding residues 19-1279 of SEQ ID NO: 1, a plasmid comprising nucleotides 55-3837 of SEQ ID NO: 2, pGX9501, INO-4800 drug product, or a biosimilar thereof. In one embodiment, the method may comprise administration of a single composition comprising an agent for the treatment of SARS-CoV-2 infection or the treatment or prevention of disease or disorder associated with SARS-CoV-2 infection and a plasmid encoding residues 19-1279 of SEQ ID NO: 1, a plasmid comprising nucleotides 55-3837 of SEQ ID NO: 2, pGX9501, INO-4800 drug product, or a biosimilar thereof.

In some embodiments, the agent for the treatment of SARS-CoV-2 infection or the treatment or prevention of disease or disorder associated with SARS-CoV-2 infection is a therapeutic agent. In one embodiment, the therapeutic agent is an antiviral agent. In one embodiment, the therapeutic agent is an antibiotic agent.

Non-limiting examples of antibiotics that can be used in combination with a plasmid encoding residues 19-1279 of SEQ ID NO: 1, a plasmid comprising nucleotides 55-3837 of SEQ ID NO: 2, pGX9501, INO-4800 drug product, or a biosimilar thereof include aminoglycosides (e.g., gentamicin, amikacin, tobramycin), quinolones (e.g., ciprofloxacin, levofloxacin), cephalosporins (e.g., ceftazidime, cefepime, cefoperazone, cefpirome, ceftobiprole), antipseudomonal penicillins: carboxypenicillins (e.g., carbenicillin and ticarcillin) and ureidopenicillins (e.g., mezlocillin, azlocillin, and piperacillin), carbapenems (e.g., meropenem, imipenem, doripenem), polymyxins (e.g., polymyxin B and colistin) and monobactams (e.g., aztreonam).

Administration as a Booster

In one embodiment, a plasmid encoding residues 19-1279 of SEQ ID NO: 1, a plasmid comprising nucleotides 55-3837 of SEQ ID NO: 2, pGX9501, INO-4800 drug product, or a biosimilar thereof is administered as a booster vaccine following administration of an initial agent or another vaccine for the treatment of SARS-CoV-2 infection or the treatment or prevention of a disease or disorder associated with SARS-CoV-2 infection, including, but not limited to COVID-19, Multisystem inflammatory syndrome in adults (MIS-A), or Multisystem inflammatory syndrome in children (MIS-C). In one embodiment, a plasmid encoding residues 19-1279 of SEQ ID NO: 1, a plasmid comprising nucleotides 55-3837 of SEQ ID NO: 2, pGX9501, INO-4800 drug product, or a biosimilar thereof is administered as a booster vaccine at least once, at least twice, at least 3 times, at least 4 times, or at least 5 times following administration of the initial agent or other vaccine for the treatment of SARS-CoV-2 infection or the treatment or prevention of a disease or disorder associated with SARS-CoV-2 infection, including, but not limited to COVID-19, Multisystem inflammatory syndrome in adults (MIS-A), or Multisystem inflammatory syndrome in children (MIS-C). In one embodiment, a plasmid encoding residues 19-1279 of SEQ ID NO: 1, a plasmid comprising nucleotides 55-3837 of SEQ ID NO: 2, pGX9501, INO-4800 drug product, or a biosimilar thereof is administered as the booster vaccine at least 8 hours, at least 12 hours, at least 16 hours, at least 20 hours, at least 24 hours, at least 36 hours, at least 48 hours, at least 60 hours, at least 72 hours, at least 4 days, at least 5 days, at least 6 days, at least 1 week at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 1 year or greater than 1 year following administration of the initial agent or other vaccine for the treatment of SARS-CoV-2 infection or the treatment or prevention of a disease or disorder associated with SARS-CoV-2 infection, including, but not limited to COVID-19, Multisystem inflammatory syndrome in adults (MIS-A), or Multisystem inflammatory syndrome in children (MIS-C).

The present invention has multiple aspects, illustrated by the following non-limiting examples.

### EXAMPLES

### Example 1 Humoral and T Cell Responses Elicited After INO-4800 Vaccination Against SARS-CoV-2 VOCs B.1.1.7, B.1.351 and P.1

INO-4800 is a SARS-CoV-2 Spike DNA-based vaccine that is delivered intradermally followed by electroporation (EP) using CELLECTRA® 2000 device and is currently undergoing clinical development. In a Phase 1 clinical trial, INO-4800 vaccination induced a balanced immune response characterized by both functional antibody and T cell responses in vaccinated subjects [Tebas, P., et al., Safety and immunogenicity of INO-4800 DNA vaccine against SARS-CoV-2: A preliminary report of an open-label, Phase 1 clinical trial. EClinicalMedicine, 2021. 31: p. 100689]. Both humoral and cellular immune responses have been shown to be important components of protection against beta-coronaviruses [Channappanavar, R., J. Zhao, and S. Perlman, T cell-mediated immune response to respiratory coronaviruses. Immunol Res, 2014. 59(1-3): p. 118-28; Sariol, A. and S. Perlman, Lessons for COVID-19 Immunity from Other Coronavirus Infections. Immunity, 2020. 53(2): p. 248-263; McMahan, K., et al., Correlates of protection against SARS-CoV-2 in rhesus macaques. Nature, 2021. 590(7847): p. 630-634.]

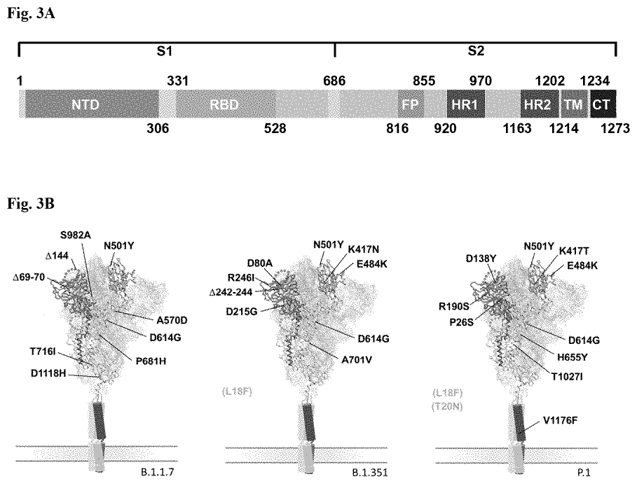

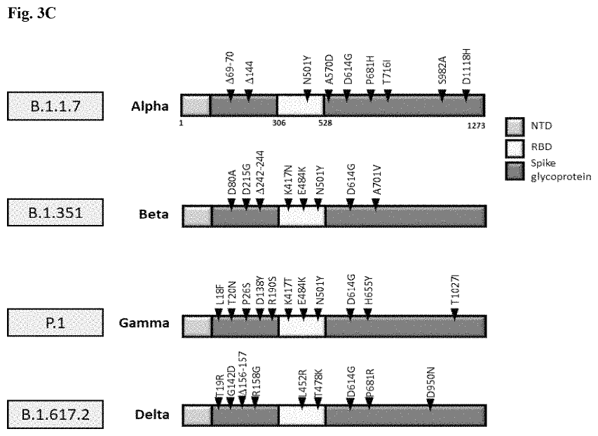

In the present study, the humoral and T cell responses elicited after INO-4800 vaccination against SARS-CoV-2 VOC, B.1.1.7, B.1.351 and P.1 (FIG. 3A) have been assessed.

Methods

Clinical Trial Subject Samples: Serum and peripheral blood mononuclear cell (PBMC) samples were acquired from participants of the phase I INO-4800 clinical trial (NCT04336410) described previously [Tebas, P., et al., Safety and immunogenicity of INO-4800 DNA vaccine against SARS-CoV-2: A preliminary report of an open-label, Phase 1 clinical trial. EClinicalMedicine, 2021. 31: p. 100689.]. The trial has since been expanded to include participants of 51-64 and 64+ years of age as separate groups in addition to the original 18-50 age group. A 0.5 mg dose group was also added. Sera from 20 subjects out of the 120 total study participants were selected for analysis on variant Spike protein binding ELISAs and variant pseudovirus neutralization assays. The samples analyzed by pseudovirus neutralization assay were collected from subjects two weeks after a third dose of INO-4800, and the samples used for other ELISA and ELISpot were collected after two doses.

Antigen Binding ELISA: Binding ELISAs were performed as described previously [Planas, D., et al., Sensitivity of infectious SARS-CoV-2 B.1.1.7 and B.1.351 variants to neutralizing antibodies. 2021: p. 2021.02.12.430472.], except different variants of SARS-CoV-2 S1+S2 proteins were used for plate coating. The S1+S2 wild-type Spike protein (Acro Biosystems #SPN-C52H8) contained amino acids 16-1213 of the full Spike protein (Accession #QHD43416.1) with R683A and R685A mutations to eliminate the furin cleavage site. The B.1.1.7, B.1.351, and P.1 S1+S2 variant proteins (Acro Biosystems #SPN-C52Hc, #SPN-C52H6, and #SPN-C52Hg, respectively) additionally contained the following proline substitutions for trimeric protein stabilization: F817P, A892P, A899P, A942P, K986P, and V987P. The B.1.1.7 protein contained the following variant-specific amino acid substitutions: HV69-70del, Y144del, N501Y, A570D, D614G, P681H, T716I, S982A, D1118H; the B.1.351 protein contained the following substitutions: L18F, D80A, D215G, R246I, K417N, E484K, N501Y, D614G, A701V; and the P.1 protein contained the following: L18F,T20N, P26S, D138Y, R190S, K417T, E484K, N501Y, D614G, H655Y, T1027I, V1176F. Assay plates were coated using 100 μL of 2 μg/mL of protein.

SARS-CoV-2 Pseudovirus Production: SARS-CoV-2 pseudovirus stocks encoding for the WT, B.1.1.7, B.1.351 or P.1 Spike protein were produced using HEK 293T cells transfected with Lipofectamine 3000 (ThermoFisher) using IgE-SARS-CoV-2 S plasmid variants (Genscript) co-transfected with pNL4-3.Luc.R-E-plasmid (NIH AIDS reagent) at a 1:8 ratio. 72 h post transfection, supernatants were collected, steri-filtered (Millipore Sigma), and aliquoted for storage at −80° C.

SARS-CoV-2 Pseudoviral Neutralization Assay: CHO cells stably expressing ACE2 (ACE2-CHOs) were used as target cells plated at 10,000 cells/well. SARS-CoV-2 pseudovirus were titered to yield greater than 30 times the cells only control relative luminescence units (RLU) after 72 h of infection. Sera from 13 INO-4800 vaccinated subjects were heat inactivated and serially diluted two folds starting at 1:16 dilution. Sera were incubated with SARS-CoV-2 pseudovirus for 90 min at room temperature. After incubation, sera-pseudovirus mixture was added to ACE2-CHOs and allowed to incubate in a standard incubator (37% humidity, 5% CO2) for 72 h. After 72 h, cells were lysed using Bright-Glo™ Luciferase Assay (Promega) and RLU was measured using an automated luminometer. Neutralization titers (ID50) were calculated using GraphPad Prism 8 and defined as the reciprocal serum dilution at which RLU were reduced by 50% compared to RLU in virus control wells after subtraction of background RLU in cell control wells.

SARS-CoV-2 Spike ELISpot assay: Peripheral mononuclear cells (PBMCs) were stimulated in vitro with 15-mer peptides (overlapping by 11 amino acids) spanning the full-length Spike protein sequence of the indicated variants. Variant peptide pools (JPT Pepmix™) included the following changes to match published deletions/mutation in each variant: B.1.1.7 variant (delta69-70, delta144, N501Y, A570D, D614G, P681H, T716I, S982A, D1118H), B.1.351 variant (L18F, D80A, D215G, delta242-244, R246I, K417N, E484K, N501Y, D614G, A701V); P.1 variant L18F, T20N, P26S, D138Y, R190S, K417T, E484K, N501Y, D614G, H655Y, T1027I, V1176F). Cells were incubated overnight with peptide pools at a concentration of 1 μg per ml per peptide in a precoated ELISpot plate, (MabTech, Human IFNγ ELISpot Plus). Cells were then washed off, and the plates were developed via a biotinylated anti-IFN-γ detection antibody followed by a streptavidin-enzyme conjugate resulting in visible spots. After plates were developed, spots were scanned and quantified using the CTL S6 Micro Analyzer (CTL) with ImmunoCapture and ImmunoSpot software. Values are shown as the background-subtracted average of measured triplicates. The ELISpot assay qualification determined that 12 spot forming units was the lower limit of detection. Thus, anything above this cutoff signal is an antigen specific cellular response.

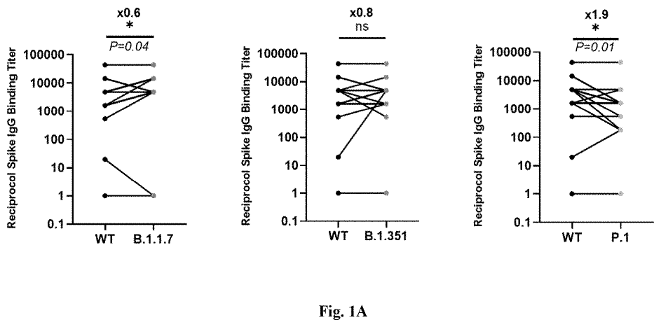

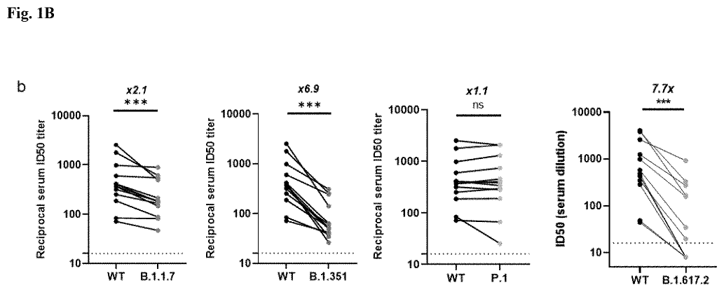

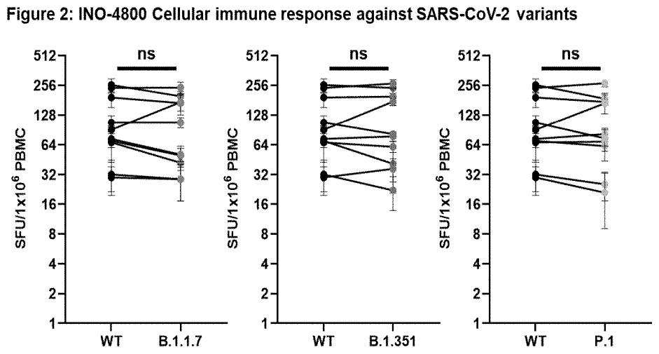

Statistical Methods: GraphPad Prism 8.1.2 (GraphPad Software, San Diego, USA) was used for graphical and statistical analysis of data sets. P values of <0.05 were considered statistically significant. A nonparametric two-tailed student t-test Wilcoxon signed-rank test was used to assess statistical significance in FIGS. 1 and 2.

Results:

Spike IgG Binding ELISA: In INO-4800 vaccinated subjects, serum IgG antibody binding titers to SARS-CoV-2 full-length Spike proteins were evaluated by ELISA using proteins specific for B.1.1.7, B.1.351, and P.1 variants (FIGS. 1A and 3A). IgG binding titers were not negatively impacted between WT and the B.1.1.7 or B.1.351 variants. An average 1.9-fold reduction was observed for the P.1 variant in subjects tested at week 8 after receiving two doses of INO-4800 (FIG. 1A).

SARS-CoV-2 Pseudoneutralization Assay: A SARS-CoV-2 pseudovirus neutralization assay was performed using sera collected from thirteen subjects two weeks after administration of a third dose of either 0.5 mg, 1 mg, or 2 mg of INO-4800 (Table 1). Neutralizing activity was detected against WT and variants B.1.1.7, B.1.351, and P.1 in the thirteen serum samples tested (FIG. 1B).

The mean ID50 titers for the WT, B.1.1.7, B.1.351 and P.1. were 643, 295, 105, and 664, respectively (Table 1). Compared to WT, there was a 2.1 and 6.9-fold reduction for B.1.1.7 and B.1.351, respectively, while there was no difference between WT and the P.1 variant. Strikingly, while the P.1 strain presents with similar RBD mutations as B.1.351 [Wang, P., et al., Increased Resistance of SARS-CoV-2 Variant P.1 to Antibody Neutralization. 2021: p. 2021.03.01.433466; Dejnirattisai, W., et al., Antibody evasion by the Brazilian P.1 strain of SARS-CoV-2. 2021: p. 2021.03.12.435194], a reduction in neutralizing activity compared to the WT strain in INO-4800 vaccinated individuals was not observed.

TABLE 1
SARS-CoV-2 pseudo neutralization ID50

| Sample ID | Dose | WT | B.1.117 | B.135 | P1 |
|---|---|---|---|---|---|
| P1 | 1 | 71 | 46 | 41 | 66 |
| P2 | 1 | 599 | 545 | 243 | 738 |
| P3 | 1 | 407 | 180 | 26 | 382 |
| P4 | 2 | 319 | 188 | 48 | 282 |
| P5 | 2 | 978 | 887 | 310 | 1292 |
| P6 | 2 | 368 | 151 | 52 | 453 |
| P7 | 1 | 372 | 185 | 33 | 406 |
| P8 | 2 | 411 | 215 | 64 | 339 |
| P9 | 2 | 251 | 167 | 60 | 297 |
| P10 | 2 | 2550 | 493 | 142 | 2079 |
| P11 | 2 | 1766 | 610 | 255 | 2087 |
| P12 | 2 | 84 | 84 | 37 | 25 |
| P13 | 0.5 | 187 | 89 | 53 | 190 |
| n | | 13 | 13 | 13 | 13 |
| Mean | | 643.3 | 295.4 | 105 | 664.3 |
| SD | | 729 | 255 | 98.87 | 706.7 |
| Range min | | 70.7 | 46.5 | 25.6 | 25.3 |
| Range max | | 2550 | 886.7 | 309.8 | 2087 |

Cellular immune responses to WT and SARS-CoV-2 Spike variants elicited by INO-4800 vaccination were compared. Peripheral blood mononuclear cells (PBMCs) isolated from ten subjects at week 8 after receiving their second dose of INO-4800 were stimulated with WT, B.1.1.7, B.1.351, P.1 (Example 1), or B.1.617.2 (Example 2). Spike peptides and cellular responses were measured by IFNγ ELISpot assay. Interestingly, similar cellular responses to WT (median=82.2 IFNγ spot-forming units [SFUs]/10⁶ PBMCs, IQR=58.9-205.3), B.1.1.7 (median=79.4, IQR=38.9-179.7), B.1.351 (median=80.0, IQR=40.0-208.6) and P.1 (median=78.3, IQR=53.1-177.8) Spike peptides were observed (FIG. 2). Between the WT and B.1.617.2, the median IFNγ spot-forming units [SFUs]/10⁶ PBMCs was 123.3 IQR=54.4-245.6 and 124.4, IQR=46.4-195.0, respectively (Example 2; FIG. 5). It is important to note that T cell responses generated by INO-4800 vaccination are consistently maintained between WT and SARS-CoV-2 variants B.1.1.7, B.1.351, and P.1. Cells stimulated with peptides against these variants generated IFNγ responses as well as cytokines associated with CD8+ cytotoxic T cell responses (data not shown).

These results show the neutralizing antibody and T cell activity measured in INO-4800 vaccinated subjects against emerging SARS-CoV-2 variants first detected in the United Kingdom, South Africa, and Brazil. The neutralization levels of INO-4800 SARS-CoV-2 Spike DNA vaccine against B.1.351 and B.1.1.7 are consistent with previous reports of subjects receiving vaccines encoding for the WT Spike protein [Wang, Z., et al., mRNA vaccine-elicited antibodies to SARS-CoV-2 and circulating variants. Nature, 2021; Stephenson, K. E., et al., Immunogenicity of the Ad26.COV2.S Vaccine for COVID-19. Jama, 2021.]. Surprisingly, despite recent reports showing a reduction in neutralizing activity against the P.1 variant [Garcia-Beltran, W. F., et al., Multiple SARS-CoV-2 variants escape neutralization by vaccine-induced humoral immunity. Cell, 2021.; Wang, P., et al., Increased Resistance of SARS-CoV-2 Variant P.1 to Antibody Neutralization. 2021: p. 2021.03.01.433466.], INO-4800 generated robust neutralizing antibodies at levels against this variant which were comparable to those against the WT strain. Taken together with the data showing the maintenance of T cell activity, the results reported in this study provide a comprehensive overview of cross-reactive cellular and humoral immune responses against SARS-CoV-2 variants for INO-4800 vaccinated individuals that may be important for protection against variant strains of SARS-CoV-2.

### Example 2 Humoral and T Cell Responses Elicited After INO-4800 Vaccination Against SARS-CoV-2 VOC B.1.617.1 and B.1.617.2

Methods

SARS-CoV-2 Pseudovirus Production: SARS-CoV-2 pseudovirus stocks encoding for the Wuhan or B.1.617.1 Spike proteins were produced using HEK 293T cells transfected with Lipofectamine 3000 (ThermoFisher) using IgE-SARS-CoV-2 Spike plasmid variants (Genscript) co-transfected with pNL4-3.Luc.R-E-plasmid (NIH AIDS reagent) at a 1:8 ratio. 72 h post transfection, supernatants were collected, steri-filtered (Millipore Sigma), and aliquoted for storage at −80° C.

SARS-CoV-2 Pseudoviral Neutralization Assay: Chinese hamster ovary (CHO) cells stably expressing ACE2 (ACE2-CHOs) were used as target cells plated at 10,000 cells/well. SARS-CoV-2 pseudovirus were titered to yield greater than 30 times the cells only control relative luminescence units (RLU) after 72 h of infection. Sera from 12 INO-4800 vaccinated subjects were heat inactivated and serially diluted two-fold starting at 1:16 dilution. Sera were incubated with SARS-CoV-2 pseudovirus for 90 min at room temperature. After incubation, sera-pseudovirus mixture was added to ACE2-CHOs and allowed to incubate in a standard incubator (37% humidity, 5% CO₂) for 72 h. After 72 h, cells were lysed using Bright-Glo™ Luciferase Assay (Promega) and RLU was measured using an automated luminometer. Neutralization titers (ID50) were calculated using GraphPad Prism 8 and defined as the reciprocal serum dilution at which RLU were reduced by 50% compared to RLU in virus control wells after subtraction of background RLU in cell control wells.

SARS-CoV-2 Spike ELISpot assay for B.1.617.2: Peripheral mononuclear cells (PBMCs) were stimulated in vitro with 15-mer peptides (overlapping by 9 amino acids) spanning the full-length Spike protein sequence of the indicated variants. The B.1.617.2 variant peptide pools included the following changes to match published deletions/mutations: T19R, (G142D), Δ156, Δ157, R158G, L452R, T478K, D614G, P681R, D950N. Cells were incubated overnight with peptide pools at a concentration of 1 μg per ml per peptide in a precoated ELISpot plate, (MabTech, Human IFNγ ELISpot Plus). Cells were then washed off, and the plates were developed via a biotinylated anti-IFN-γ detection antibody followed by a streptavidin-enzyme conjugate resulting in visible spots. After plates were developed, spots were scanned and quantified using the CTL S6 Micro Analyzer (CTL) with ImmunoCapture and ImmunoSpot software. Values are shown as the background-subtracted average of measured triplicates. The ELISpot assay qualification determined that 12 spot forming units was the lower limit of detection. Thus, anything above this cutoff signal is an antigen specific cellular response.

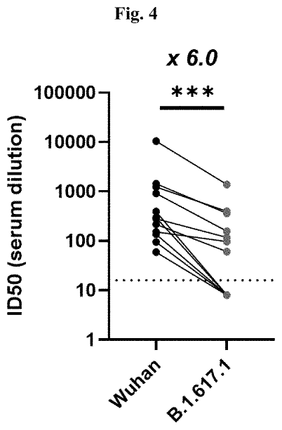

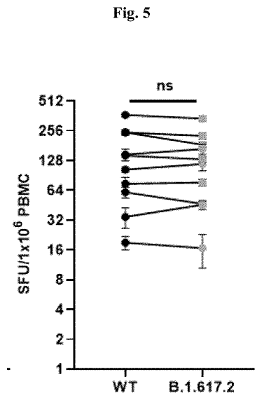

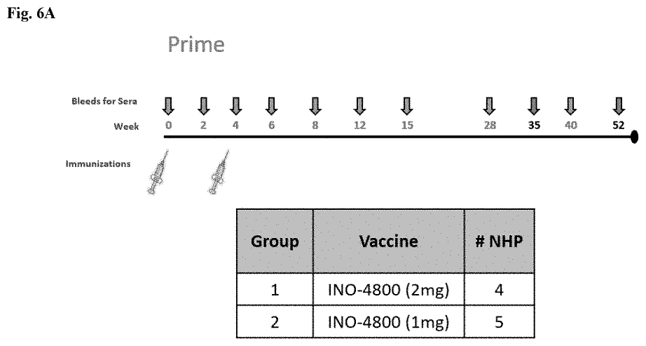

Statistical Methods: GraphPad Prism 8.1.2 (GraphPad Software, San Diego, USA) was used for graphical and statistical analysis of data sets. P values of <0.05 were considered statistically significant. A nonparametric two-tailed student t-test Wilcoxon signed-rank test was used to assess statistical significance in FIGS. 4 and 5.

Results

A SARS-CoV-2 pseudovirus neutralization assay was performed using sera collected from twelve subjects two weeks after administration of a second dose of INO-4800 (6 weeks post-first immunization). Neutralizing activity was detected against Wuhan pseudovirus in all samples tested. For the B.1.617.1 variant, 7 out of 12 samples showed cross-neutralizing activity, with a reduction of 6-fold in neutralization compared to the WT pseudovirus (FIG. 4). The mean ID50 titers for the Wuhan and B.1.617.1 were 1304 and 217, respectively. Seven of twelve samples showed cross-neutralizing activity above the LOD for B.1.617.2 (FIG. 1B). Between WT and B.1.617.2, the mean ID50 titer was 1251 and 162, respectively. Compared to WT, there was a 7.7-fold reduction for B.1.617.2.

Cellular immune responses to Wuhan and SARS-CoV-2 Spike variants elicited by INO-4800 vaccination were compared. Peripheral blood mononuclear cells (PBMCs) isolated from ten subjects at week 8 after receiving their second dose of INO-4800 were stimulated with Wuhan or B.1.617.2 Spike peptides and cellular responses were measured by IFNγ ELISpot assay. Strikingly similar cellular responses to Wuhan (median=123.3 IFNγ spot-forming units [SFUs]/10⁶ PBMCs, IQR=54.4-245.6) and B.1.617.2 (median=124.4, IQR=46.4-195.0) Spike peptides (FIG. 5) were observed. T cell responses are consistently maintained between Wuhan and B.1.617.2 variant.

### Example 3 Enhanced Immunity to SARS-CoV-2 Variants of Concern Following Homologous Prime-Boost Vaccination in Nonhuman Primates

This example evaluates the immunogenicity of a prime-boost regimen in nonhuman primates. Rhesus macaques received primary immunization with INO-4800, a first-generation DNA vaccine matched to SARS-CoV-2 Spike protein of the original strain and currently in clinical development. One year later, the immunized animals were randomized and received homologous boost with INO-4800. Following the boost, all animals showed significantly increased levels of functional antibody responses with neutralizing and ACE2 blocking activity against multiple SARS-CoV-2 VOCs. These data indicate homologous prime-boost strategies with the INO-4800 DNA vaccine enhances broad humoral responses against emerging SARS-CoV-2 variants.

Materials & Methods

Animals and Immunizations. All rhesus macaque experiments were approved by the Institutional Animal Care and Use Committee at Bioqual (Rockville, Md.), an Association for Assessment and Accreditation of Laboratory Animal Care (AAALAC) International accredited facility. Nine Chinese rhesus macaques (five males, and four females roughly 4 years of age, ranging from 4.48 kg-8.50 kg) were randomized prior to injection and were initially immunized with one or two 1 mg injections of the SARS-CoV-2 DNA vaccine INO-4800 drug product at weeks 0 and 4 by a minimally invasive intradermal electroporation (ID-EP) administration using the CELLECTRA 2000® Adaptive Constant Current Electroporation Device with a 3P array (Inovio Pharmaceuticals). Approximately one year post prime immunization, a randomized subset of four study animals received a boost immunization at 1 mg per dose of INO-4800 drug product by ID-EP administration. Sera samples collected at each timepoint were used to evaluate binding titers, pseudovirus neutralization, intracellular cytokine staining (ICS), and ACE2 blocking activity and to isolate peripheral blood mononuclear cells (PBMC) and serum.

Peripheral Blood Mononuclear Cell Isolation and IFN-γ Enzyme-Linked Immunospot (ELISpot)

Blood was collected from each study animal into sodium citrate cell preparation tubes (CPT, BD Biosciences). The tubes were centrifuged to separate plasma and lymphocytes, according to the manufacturer's protocol. Samples from the prime immunization were transported by same-day shipment on cold-packs from Bioqual to The Wistar Institute, and boost samples were shipped overnight to Inovio Pharmaceuticals for PBMC isolation. PBMCs were washed, and residual red blood cells were removed using ammonium-chloride-potassium (ACK) lysis buffer. Cells were counted using a ViCell counter (Beckman Coulter) and resuspended in RPMI 1640 (Corning), supplemented with 10% fetal bovine serum (Seradigm), and 1% penicillin/streptomycin (Gibco). Fresh cells were then plated for IFNγ ELISpot assay to detect cellular responses.

Monkey IFN-γ ELISpotPro plates (Mabtech, Sweden, Cat#3421M-2APW-10) were prepared according to the manufacturer's protocol. Freshly isolated PBMCs were added to each well at 200,000 cells per well in the presence of either 1) SARS-CoV-2-specific peptide pools, 2) R10 with DMSO (negative control), or 3) anti-CD3 positive control (Mabtech, 1:1000 dilution), in triplicate. Plates were incubated overnight at 37° C., 5% CO₂, then after a minimum incubation of 18 hours, plates were developed according to the manufacturer's protocol. Spots were imaged using a CTL Immunospot plate reader and antigen-specific responses determined by subtracting the R10-DMSO negative control wells from the wells stimulated with peptide pools.

Antigen-binding ELISA. Nunc plates were coated with 1 ug/mL recombinant SARS-CoV-2 S1+S2 spike proteins and binding titers were determined after background subtraction of animals vaccinated with mock vector. For prime immunization samples, ninety-six well immunosorbent plates (NUNC) were coated with 1 μg/mL recombinant SARS-CoV-2 S1+S2 ECD protein (Sino Biological 40589-V08B1), S1 protein (Sino Biological 40591-V08H), S2 protein (Sino Biological 40590-V08B), or receptor-binding domain (RBD) protein (Sino Biological 40595-V05H) in PBS overnight at 4° C. For boost samples, ELISA half-area plates were coated with 1 μg/mL recombinant spike Wild-Type spike protein, Alpha (B.1.1.7), Beta (B.1.351), Gamma (P.1), Delta (B.1.617.2), and Omicron (B.1.1.529) full length spike variant proteins (Acro Biosystems #SPN-C52H8, #SPN-C52Hc, #SPN-C52Hg, #SPN-C52He, and #SPN-C52Hz, respectively). Secondary antibodies included IgG (Bethyl #A140-202P) at 1:50,000, IgG2A (Abcam #ab98698), and IgG1 (Abcam #ab98693) at 1:10,000 dilution. Plates were washed three times with PBS+0.05% Tween20 (PBS-T) and blocked with 3% FBS in PBS-T for 2 hours at room temperature (RT). Sera from vaccinated macaques were serially diluted in PBS-T+1% FBS, added to the washed ELISA plates, and then incubated for 2 hours at RT. Plates were then washed and incubated with an anti-monkey IgG conjugated to horseradish peroxidase (Bethyl A140-202P) 1 hour at RT. Within 30 minutes of development, plates were read at 450 nm using a Biotek Synergy2 plate reader.

Pseudovirus Neutralization Assay

SARS-CoV-2 pseudovirus stocks encoding for the wild-type (WT), Alpha (B.1.1.7), Beta (P.1), Gamma (B.1.351), Delta (B.1.617.2), or Omicron (B.1.1.529) Spike protein were produced using HEK 293T cells transfected with Lipofectamine 3000 (ThermoFisher) using IgE-SARS-CoV-2 S plasmid variants (Genscript) co-transfected with pNL4-3.Luc.R-E-plasmid (NIH AIDS reagent). To assess neutralizing activity of serum antibodies, CHO cells stably expressing ACE2 (ACE2-CHOs—Creative Biolabs) were used as target cells at 10,000 cells/well. Sera was heat inactivated and serially diluted prior to incubation with the different SARS-CoV-2 variant pseudoviruses. After a 90-minute incubation, sera-pseudovirus mixture was added to ACE2-CHOs, then 72 hours later, cells were lysed using Bright-Glo™ Luciferase Assay (Promega) and RLU was measured using an automated luminometer. Neutralization titers (ID₅₀) were calculated using GraphPad Prism 8 and defined as the reciprocal serum dilution that is reduced by 50% compared to the signal in the infected control wells.

Meso Scale Discovery ACE2 Blocking Assay

Functional antibody responses were also assessed based on inhibition of ACE2 blocking to SARS-CoV-2 Spike protein (and VOC Spike proteins). For these assays, the Meso Scale Discovery (MSD) V-PLEX SARS-CoV-2 ACE2 Neutralization Kit, Panels 5 and 14, were used according to the manufacturer's instructions with the MSD Sector S 600 instrument. Briefly, MSD plates containing SARS-CoV-2 Spike proteins (wildtype, B.1.1.7, B.1.351, P.1, and B.1.617.2) were blocked, washed, and incubated with sera from vaccinated animals at a 1:27 dilution. Plates were then washed and incubated with SULFO-TAG ACE2 and developed according to the manufacturer's protocol. Functional antibody activity was measured as % inhibition of binding of SULFO-TAG ACE2 to Spike protein.

Peripheral Blood Mononuclear Cell (PBMC) Isolation and Intracellular Cytokine Staining (ICS)

Blood was collected from each study animal into sodium citrate cell preparation tubes (CPT, BD Biosciences). The tubes were centrifuged to separate plasma and lymphocytes, according to the manufacturer's protocol. Samples from the prime immunization were transported by same-day shipment on cold-packs from Bioqual to The Wistar Institute, and boost samples were shipped overnight to Inovio Pharmaceuticals for PBMC isolation. PBMCs were washed, and residual red blood cells were removed using ammonium-chloride-potassium (ACK) lysis buffer. Cells were counted using a ViCell counter (Beckman Coulter) and cryopreserved in 90% fetal bovine serum (FBS)/10% dimethyl sulfoxide (DMSO). For ICS assays, cells were thawed in RPMI 1640 (Corning), supplemented with 10% fetal bovine serum (Seradigm), and 1% penicillin/streptomycin (Gibco).

For ICS, following an overnight rest at 37° C., PBMCs (1×10⁶/sample) were added to each well and stimulated with either 1) SARS-CoV-2-specific peptide pools, 2) R10 with DMSO (negative control), or 3) eBioscience Cell Stimulation Cocktail containing phorbol 12-myristate 13-acetate (PMA) and ionomycin (Invitrogen, 1:1000 dilution) in the presence of GolgiStop™ and GolgiPlug™ (Invitrogen) and anti-CD28/CD49d. Plates were incubated for 6 hours at 37° C., 5% CO₂, washed, and then stained using an antibody cocktail containing anti-CD3 APC-Cy7, anti-CD4 PerCP-Cy5.5, anti-CD8 BV786, and LIVE/DEAD Fixable Aqua Dead Cell Stain (Invitrogen). Cells were then fixed, permeabilized (eBioscience Foxp3/Transcription Factor Fixation/Permeabilization Kit; ThermoFisher), and stained for intracellular cytokines using an antibody cocktail containing anti-IFNγ BV605, anti-IL-2 BV650, and anti-TNFα APC-R700. Cells were then washed, resuspended and acquired on a BD FACS Celesta. Data were analyzed using FlowJo™ v10.7 Software (BD Life Sciences).

Results

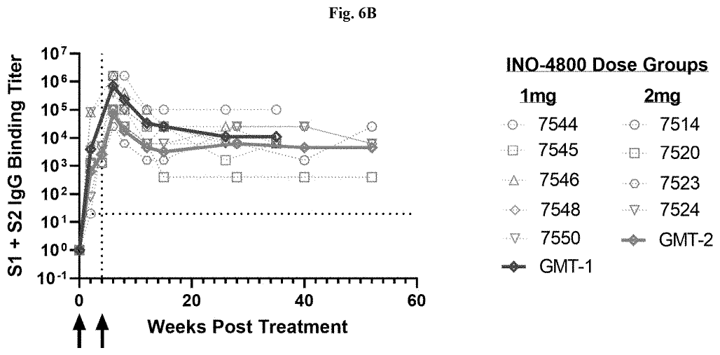

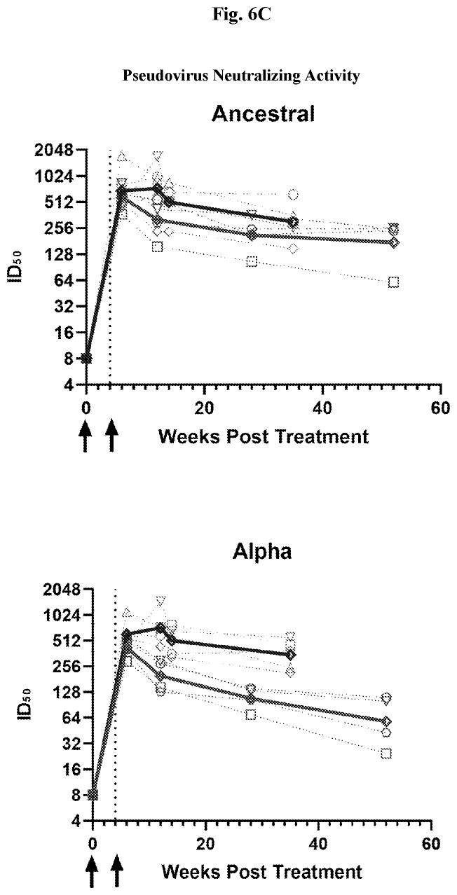

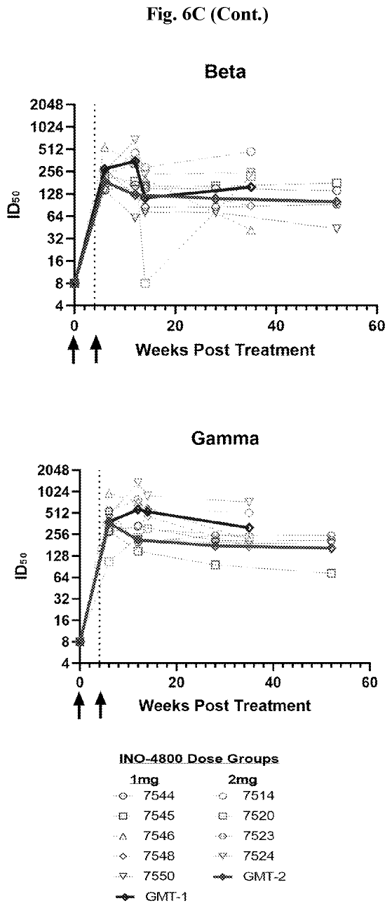

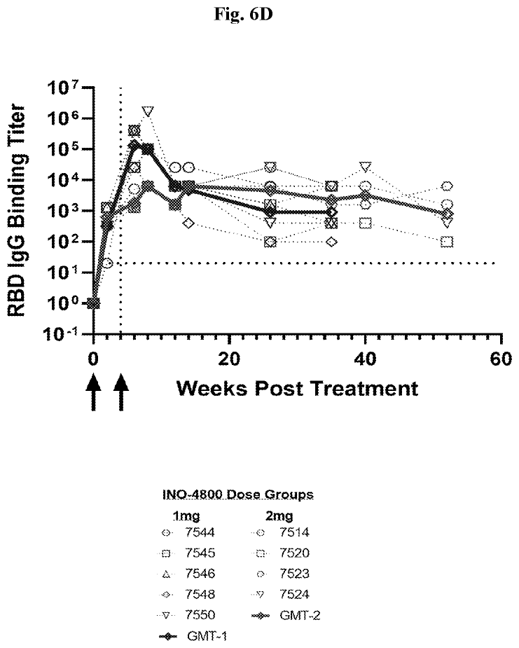

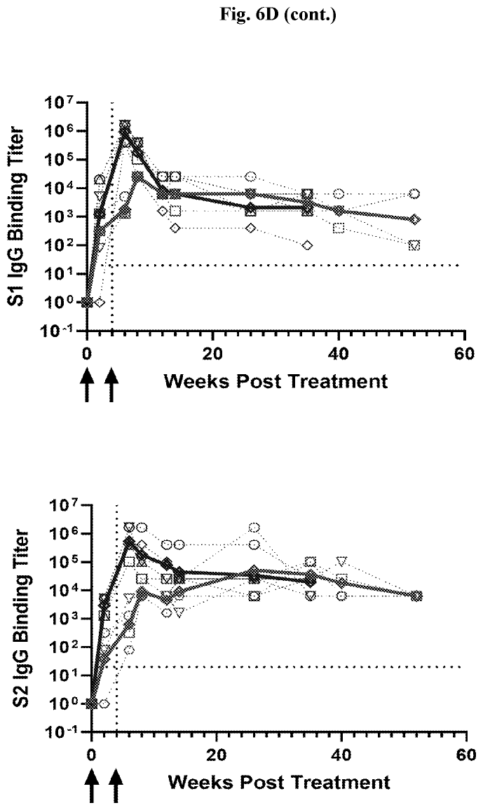

Durability following INO-4800 primary immunization. Initial studies investigated the durability of immune responses in non-human primates (NHPs) primed with INO-4800. NHPs were immunized at week 0 and 4 with either a 1 mg or 2 mg dose of INO-4800, and blood was collected over the course of one year (FIG. 6A). It should be noted that, for FIGS. 6A-6D, the NHPs were initially treated on staggered schedules, and therefore the data from the prime immunization portion of the study show collected data points for NHP IDs #7544, 7545, 7546, 7548, 7550 terminating at Week 35 and for others, IDs #7514, 7520, 7523, 7524, terminating at Week 52. An enzyme-linked immunosorbent assay (ELISA) was used to measure levels of binding antibodies in the serum. Peak antibody titers were observed at week 6 with a geometric mean endpoint titer of 258,032, two weeks following the second immunization (FIG. 6B). Detectable levels of binding antibodies persisted in the serum for the duration of the study, and at the final timepoint prior to boosting, the 1 mg dose group had geometric mean endpoint titers of 11143 for the S1+S2 ECD. The 2 mg dose group had geometric mean endpoint titers of 4525 for the Sl+S2 ECD. Similar trends were also observed in the levels of binding antibodies against the SARS-CoV-2 S1, SARS-CoV-2 S2 and RBD proteins (FIG. 6D).

Functional antibody responses were measured in a pseudovirus neutralization assay against the SARS-CoV-2 ancestral, Alpha, Beta and Gamma variants of concern (VOCs) which were in circulation during this time period. Immunization with INO-4800 resulted in the induction of neutralizing antibodies that were increased over baseline for all VOCs (FIG. 6C). SARS-CoV-2 VOC neutralizing antibody responses were durable and remained elevated over baseline at the last collected timepoint, with the 1 mg dose group having a geometric mean titer (GMT) of 301 against ancestral SARS-CoV-2, 349 for Alpha, 158 for Beta, and 317 for Gamma. NHP #7545 showed reduced neutralizing activity at Week 14 for Beta which was attributed to sampling error during plating. The 2 mg dose group had a GMT of 174.6 for the wild-type variant, 58.2 for Alpha, 100.3 for Beta, and 164.2 for Gamma. Together, these data illustrate that the primary INO-4800 vaccination schedule induced SARS-CoV-2 specific antibodies harboring neutralizing activity that were maintained over the period of 35-52 weeks.

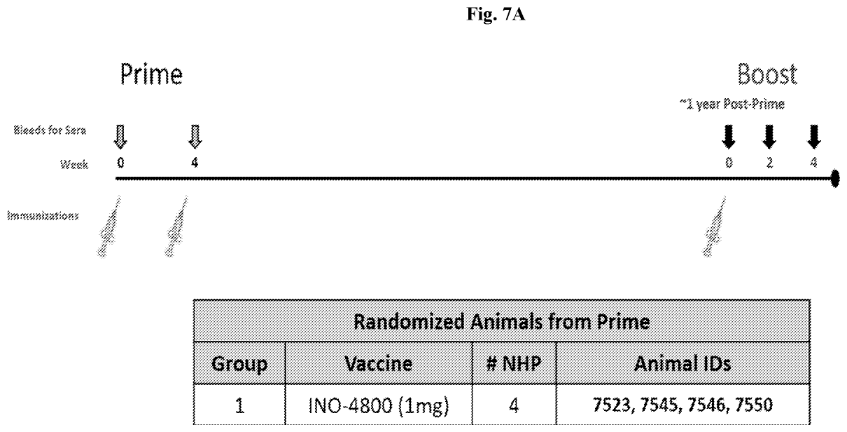

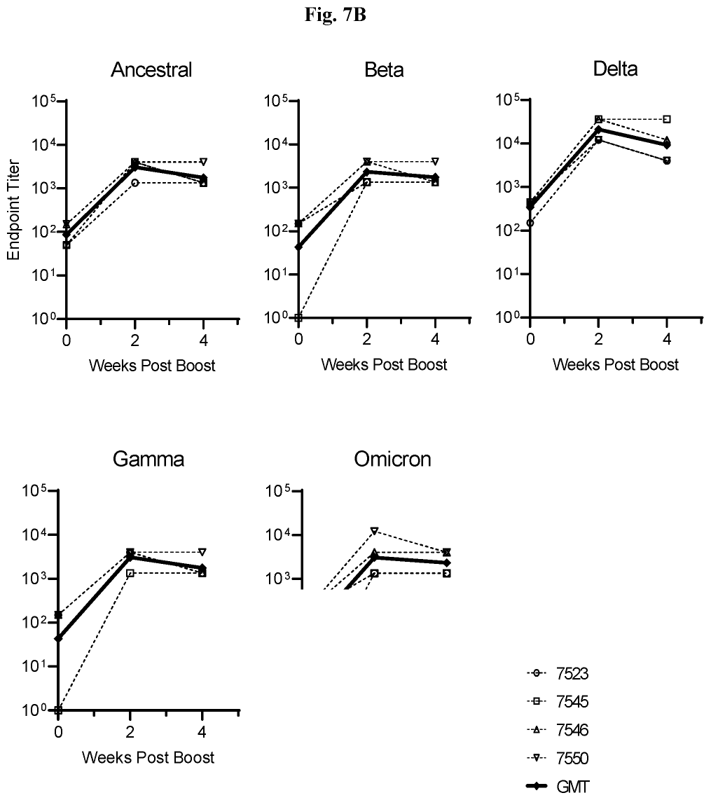

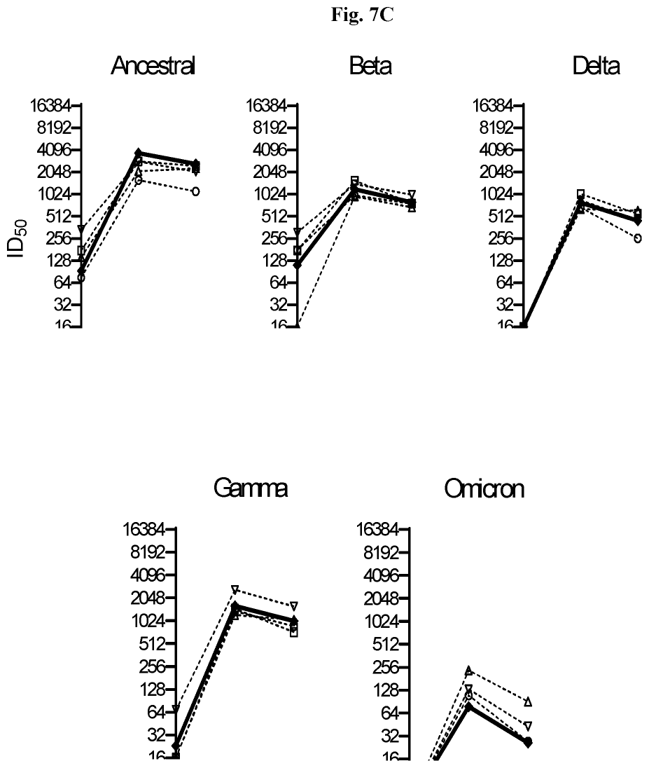

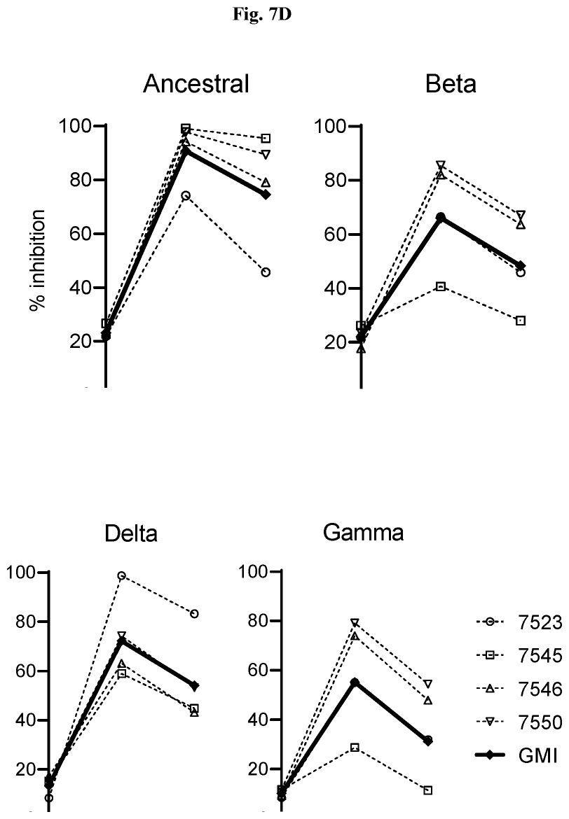

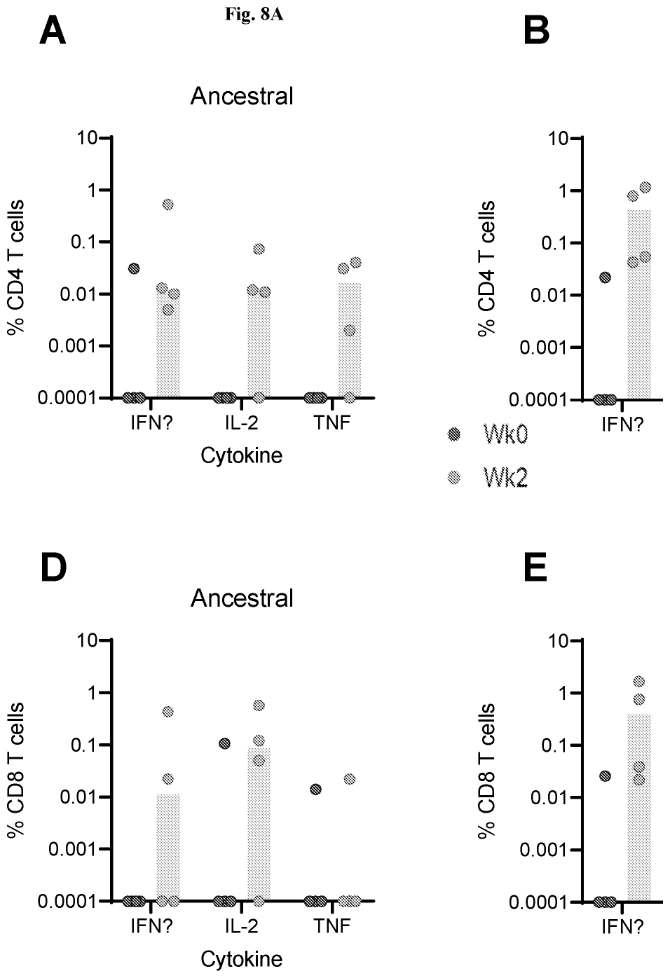

Humoral responses following delivery of INO-4800. INO-4800 was evaluated as a booster vaccine. Four of the same rhesus macaques that were initially primed with INO-4800 were boosted with INO-4800, homologous to the original vaccine. Rhesus macaques #7545, 7546, and 7550 were boosted 43 weeks after the initial vaccination while NHP #7523was boosted at 64 weeks after the initial vaccination (FIG. 7A).

The homologous boost with INO-4800 resulted in the induction of antibody titers at two weeks post-boost that were increased over pre-boost levels (FIG. 7B). Increases in binding antibody levels showed similar patterns against the ancestral, Beta, Delta, Gamma, and Omicron Spike proteins, with GMTs of 87, 43, 342, 43, and 43, respectively, pre-boost and 3077, 2338, 21044, 3077, and 3077, respectively, post-boost. Binding titers against any of the variants were not significantly different between INO-4800-boosted animals at either Week 2 or Week 4.

Neutralizing activity against the ancestral, Beta, Delta, Gamma, and Omicron variants was assessed by a pseudovirus neutralization assay, which revealed increased neutralizing antibody responses against all SARS-CoV-2 variants in animals boosted with INO-4800 (FIG. 7C). The GMTs at Week 2 for the NHPs after the homologous INO-4800 boost were 2286.2, 1199.3, 785.6, 1596.1, and 78.3 against the ancestral, Beta, Delta, Gamma, and Omicron pseudoviruses, respectively. INO-4800-boosted animals did not show a significant difference in neutralization of the ancestral, Beta, and Omicron pseudoviruses at either Week 2 or Week 4. As an additional readout of functional antibody responses, ACE2/SARS-CoV-2 Spike interaction blocking activity of serum antibodies was measured using a Meso Scale Discovery (MSD) assay, by quantifying the level of inhibition of ACE2 binding to a panel of variant SARS-CoV-2 Spike proteins. In line with the pseudovirus neutralization data, all animals showed an increase in the level of functional anti-SARS-CoV-2 antibodies in their serum following the boost immunization (FIG. 7D).

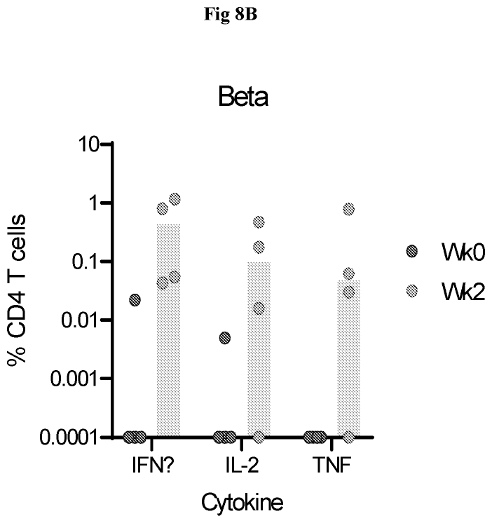

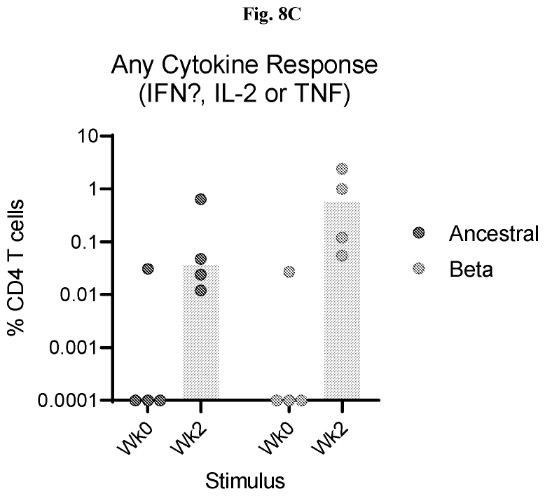

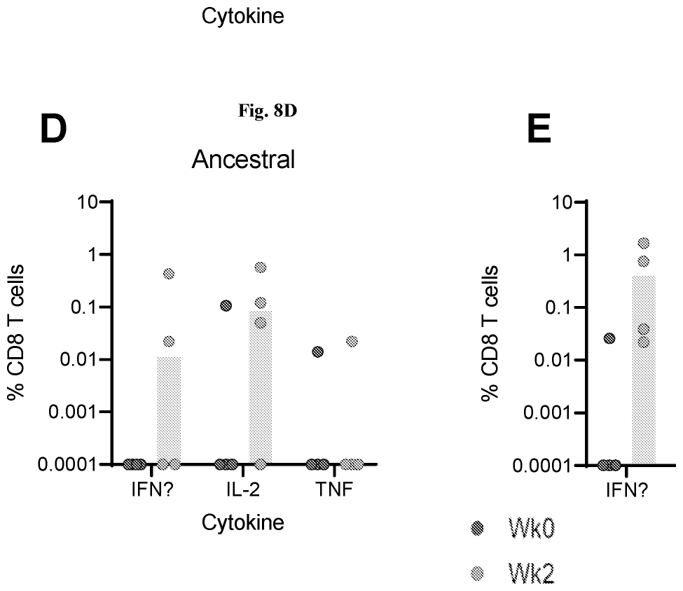

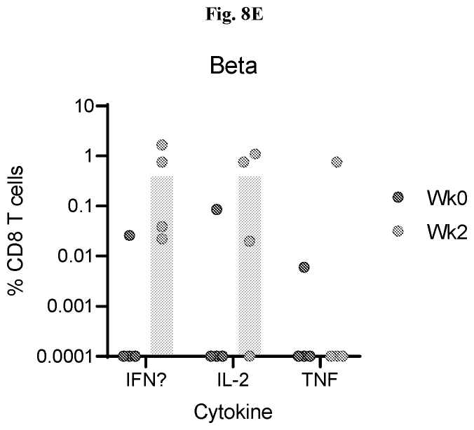

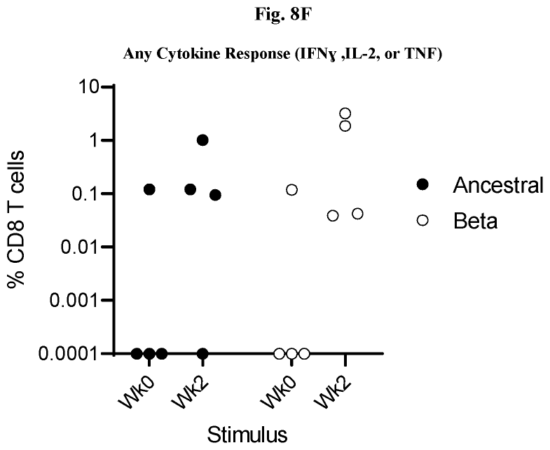

Induction of cellular responses by INO-4800. Intracellular cytokine staining (ICS) was performed on peripheral blood mononuclear cells (PBMCs) stimulated with peptides matching the ancestral or Beta SARS-CoV-2 Spike proteins to evaluate cellular responses in rhesus macaques boosted with INO-4800. Antigen-specific CD4 and CD8 T cell responses were observed in animals boosted with either vaccine (FIGS. 8A-8F). The magnitude of cellular responses was generally greater at 2 weeks post-boost relative to pre-boost levels and showed that boosting with INO-4800 induced CD4 T cell responses that were maintained across the ancestral and Beta variants (FIGS. 8A-8C). Phenotypic analysis of the CD4 T cell responses at Week 2 showed IFNγ secretion in all animals and IL-2 and TNF secretion in 3 of 4 animals (FIGS. 8A, 8B). Similar responses were observed in the CD8 compartment at Week 2, showing secretion of IFNγ (2 of 4 animals for ancestral and 4 of 4 animals for Beta) and IL-2 (3 of 4 animals for each VOC) (FIGS. 8D-8F).

### Example 4 Maintenance of T Cell Activity Against the SARS-CoV-2 Omicron Variant

Methods:

Clinical Trial Subject Samples. Serum and PBMC samples were acquired from participants of the phase I INO-4800 clinical trial (NCT04336410) described previously (Tebas, P., et al., EClinicalMedicine, 2021. 31: p. 100689). The trial includes participants in an 18-50 age group, a 51-64 age group, and a 64+ years age group. The trial also includes dose groups of 0.5 mg, 1 mg, or 2 mg. Serum samples were also acquired from participants of the phase II clinical trial (NCT04642638) which evaluated a two-dose regimen (1 mg or 2 mg) of INO-4800 (Mammen, M. P., et al., 2021, medRxiv, 2021.05.07.21256652). Sera from 10 subjects out of the 120 total study participants were selected for analysis of the Omicron Spike and RBD protein binding ELISAs and pseudovirus neutralization assays. The samples analyzed by ELISA and pseudovirus neutralization assay were collected from subjects two weeks after two doses of INO-4800.

Antigen Binding ELISA. Binding ELISAs were performed as described in Example 1. The S1+S2 wild-type Spike protein (Acro Biosystems #SPN-C52H9) contained amino acids 16-1213 of the full Spike protein (Accession #QHD43416.1) with R683A and R685A mutations to eliminate the furin cleavage site and contained the following proline substitutions for trimeric protein stabilization: F817P, A892P, A899P, A942P, K986P, and V987P. The Omicron variant Spike protein (Acro Biosystems #SPN-C52Hz) contained the same amino acid substitutions for trimeric protein stabilization and additionally contained the following Omicron-specific mutations: A67V, HV69-70del, T95I, G142D, VYY143-145del, N211del, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. The Omicron RBD variant protein (Acro Biosystems #SPD-C522e r, Accession #QHD43416.1) contained amino acids Arg 319-Lys 537 with the following omicron-specific mutations: G339D, S371L, S373P, S375F, K417N, N440K, G446S, S477N, T478K, E484A, Q493R, G496S, Q498R, N501Y, and Y505H. Assay plates were coated with 0.2 μg of protein in a volume of 100 μL. Optical densities at 450 nm with background subtraction at OD₆₅₀ were reported for clinical study samples that were diluted 1/100 for the full-spike assay and 1/20 for the RBD assay.

SARS-CoV-2 Pseudovirus Production. SARS-CoV-2 pseudovirus stocks expressing the WT and Omicron Spike proteins were produced using HEK 293T cells transfected with Lipofectamine 3000 (ThermoFisher) using IgE-SARS-CoV-2 Spike plasmids (Genscript) co-transfected with pNL4-3.Luc.R-E-plasmid (NIH AIDS reagent) at a 1:8 ratio. Supernatants were collected 72 h after transfection, sterile filtered (Millipore Sigma), and aliquoted for storage at −80° C.

SARS-CoV-2 Pseudoviral Neutralization Assay. CHO cells stably expressing ACE2 (ACE2-CHOs) and plated at 7,000 cells/well were used as target cells. SARS-CoV-2 pseudovirus were titrated to yield>30 times the relative luminescence units (RLU) versus the cells-only control after 72 h of infection. Sera from 10 INO-4800-vaccinated subjects were heat inactivated and serially diluted two-fold starting at 1:8 dilution. Sera were incubated with SARS-CoV-2 pseudovirus for 90 min at room temperature. After incubation, the sera-pseudovirus mixture was added to ACE2-CHOs and allowed to incubate in a standard incubator (37% humidity, 5% CO₂) for 72 h, after which the cells were analyzed using the Britelite plus Reporter Gene (PerkinElmer™) assay and RLU was measured using an automated luminometer. Neutralization titers (ID₅₀) were calculated using GraphPad Prism 8 and defined as the reciprocal serum dilution at which RLU were reduced by 50% compared to RLU in virus control wells after subtraction of background RLU in cell control wells.

SARS-CoV-2 Spike ELISpot assay. Peripheral blood mononuclear cells (PBMCs) were stimulated in vitro with 15-mer peptides (overlapping by 10 amino acids) spanning the full-length Spike protein sequence for the ancestral and Omicron variants, respectively, which were provided as megapools by the Sette lab at La Jolla institute for Immunology (Tarke, A., et al., 2021: p. 2021.12.28.474333). Cells were incubated overnight with peptide megapools at a final concentration of 1 μg/mL in a precoated ELISpot plate (MabTech, Human IFNγ ELISpot Plus). Cells were processed and plates were developed as previously described (Tebas, P., et al., EClinicalMedicine, 2021. 31: p. 100689). Values are shown as the background-subtracted average of measured triplicates. The ELISpot assay qualification determined that 12 SFU/10⁶ was the lower limit of detection; anything above this cutoff is therefore considered an antigen-specific cellular response.

SARS-CoV-2 Spike flow cytometry assay. PBMCs were also used for Intracellular Cytokine Staining (ICS) analysis. One million PBMCs in 200 mL complete RPMI media were stimulated for six hours (37° C., 5% CO₂) with DMSO (negative control), PMA and Ionomycin (positive control, 100 ng/mL and 2 mg/mL, respectively), or with the indicated peptide megapools (1 μg/mL). After the first hour of stimulation, Brefeldin A and Monensin (BD GolgiStop and GolgiPlug, 0.001% and 0.0015%, respectively) were added to block secretion of expressed cytokines. Cells were then moved to 4° C. overnight and the following day, staining was conducted as previously described (Tebas, P., et al., EClinicalMedicine, 2021. 31: p. 100689). Data was analyzed using the FlowJo v10 software (BD).

Statistical Methods. GraphPad Prism 8.1.2 (GraphPad Software, San Diego, USA) was used for graphical and statistical analysis of data sets. P values of <0.05 were considered statistically significant. A nonparametric two-tailed student t-test Wilcoxon signed-rank test was used to assess statistical significance in FIGS. 1 and 2.

Results:

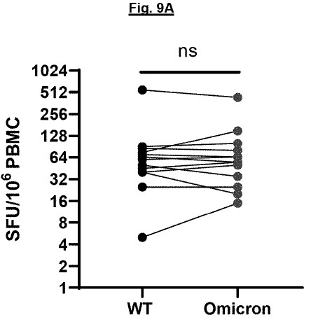

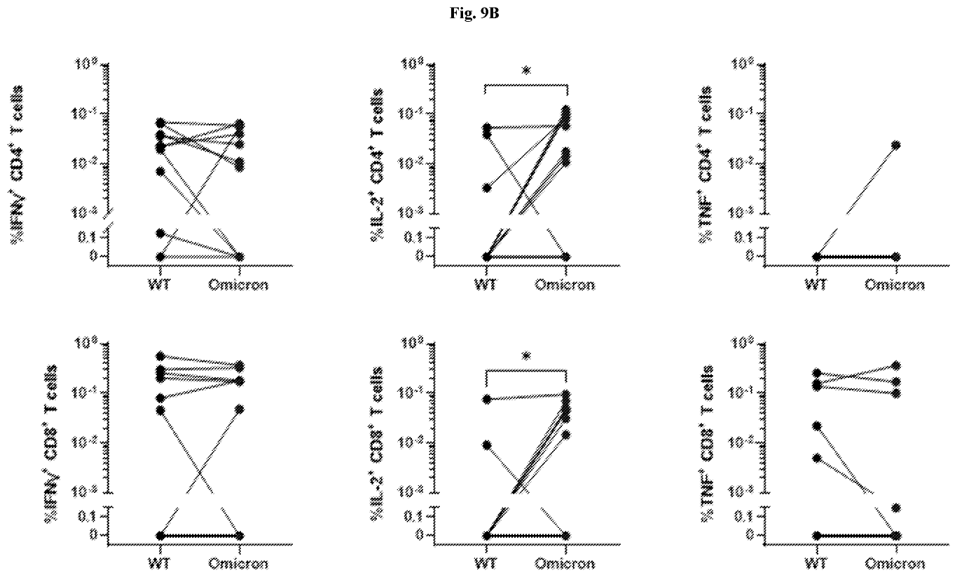

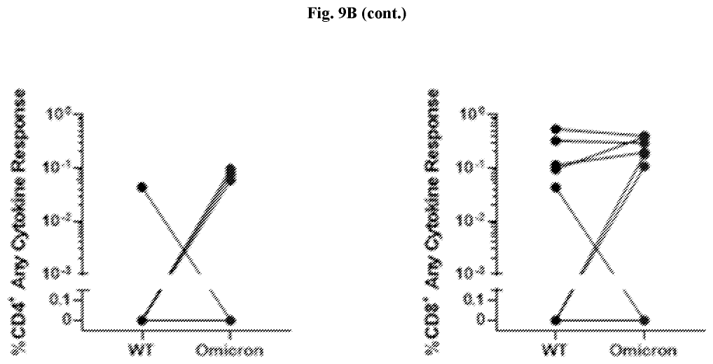

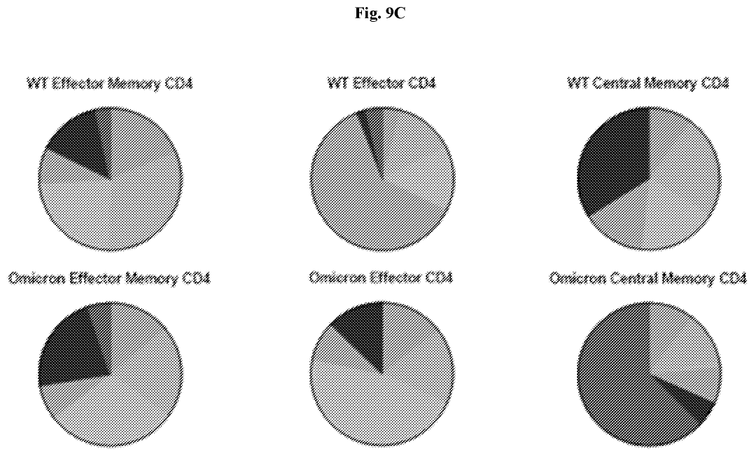

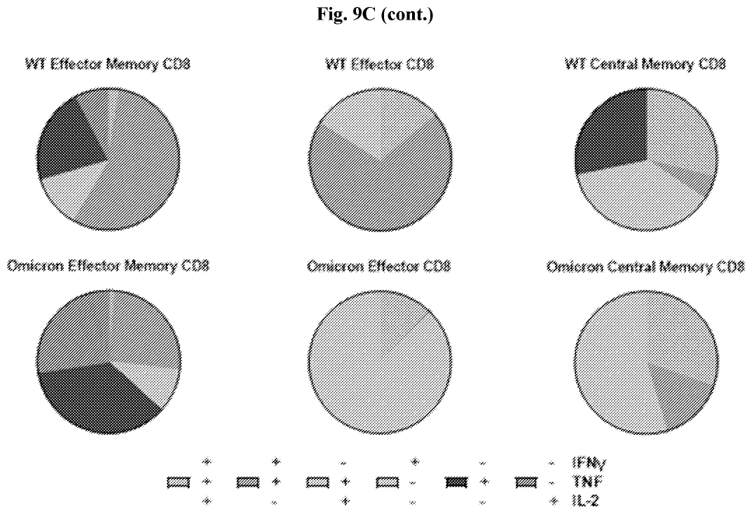

Humoral and cellular immune responses induced against the Omicron variant have been evaluated by examining binding antibody responses in ten subjects previously vaccinated with INO-4800 (1.0 mg, n=3; 2.0 mg, n=7), neutralizing antibody responses in twelve subjects (0.5 mg, n=1; 1.0 mg, n=3; 2.0 mg, n=8), and T cell responses in thirteen subjects (0.5 mg, n=4; 1.0 mg, n=4 and 2.0 mg, n=5). Peripheral blood mononuclear cells (PBMCs) isolated from thirteen subjects 8 weeks (Week 12) after their second dose of INO-4800 were stimulated with ancestral (WT) or Omicron spike peptides (Tarke, A., et al., SARS-CoV-2 vaccination induces immunological memory able to cross-recognize variants from Alpha to Omicron. 2021: p. 2021.12.28.474333), and cellular responses were measured by IFNγ ELISpot assay. Similar levels of T cell responses to WT (mean=92.3, IFNγ spot-forming units [SFUs]/10⁶ PBMCs, IQR=40-80) and Omicron (mean=88.5, IQR=30-90) spike peptides were observed (FIGS. 9A-9C).

The impact of the Omicron variant on CD4 and CD8 T cell responses was assessed by intracellular cytokine staining and flow cytometry. Stimulated PBMCs isolated from eleven subjects (selected from the subset of Phase 1 patients described above in the IFNγ ELISpot assay) prior to vaccination (Week 0) and 6 weeks (Week 12) after the second dose of INO-4800 were assayed for IFNγ, IL-2, and TNFα production. The data illustrates induction of WT and Omicron-specific responses in both the CD4 and CD8 T cell compartments. While similar frequencies of IFNγ- and TNFα-producing CD4 and CD8 T cells were observed, a higher frequency of IL-2-specific responses was observed with the Omicron variant compared to WT in both the CD4 and CD8 compartments (mean Omicron response: CD4+—0.051% and CD8+—0.062% and ancestral: CD4+—0.012% and CD8+—0.003%). Further analysis of effector memory (CCR7−CD45RA−) and central memory (CCR7+ CD45RA−) T cell populations revealed higher proportions of IL-2-producing CD4 and CD8 central memory T cells targeting Omicron as compared to WT peptides.

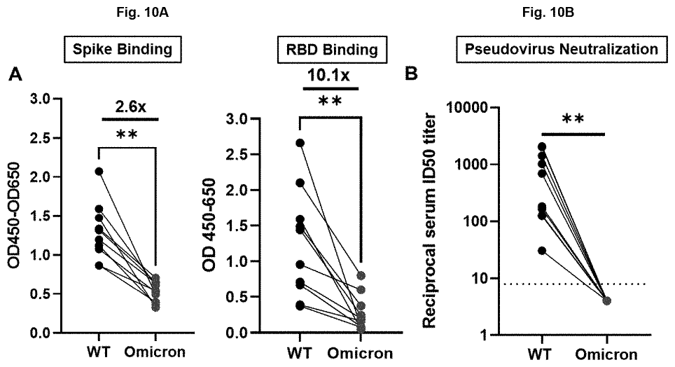

Serum IgG antibody binding titers to SARS-CoV-2 Omicron full-length Spike and RBD proteins were evaluated by ELISA. Compared to the ancestral full-length spike protein, a 2.6-fold reduction was observed for the Omicron variant spike protein in subjects tested at week 6, two weeks following the second dose of INO-4800 (FIG. 10A). With the Omicron RBD protein, a 10-fold reduction was observed in the same subjects (FIG. 10A). Similar fold reductions were observed in sera from convalescent subjects (collected in early 2020). Neutralizing activity was assessed in a SARS-CoV-2 pseudovirus neutralization assay using sera collected from subjects two weeks after a second dose of INO-4800. Neutralizing activity was detected against WT in all samples, while activity against Omicron was below the limit of detection (FIG. 10B).

Conclusion. Significant reductions in neutralization activity and levels of binding antibodies to the Omicron variant in serum collected from vaccinated individuals were observed (FIGS. 10A and 10B). Conversely, INO-4800-vaccinated subjects maintained T cell responsiveness towards Omicron, as evidenced by similar levels of functional CD4+ and CD8+ T cells compared to those elicited by the ancestral spike protein (FIGS. 9A-9C). The data showed induction of central memory T cells that exhibited a higher degree of IL-2 production for the Omicron variant compared to ancestral SARS-CoV-2.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

| SEQUENCES | |
|---|---|
| SARS-CoV-2 Consensus Spike Antigen amino acid insert | |
| sequence of pGX9501 (SEQ ID NO 1) (IgE leader sequence | |
| underlined): | |
| 1 | MDWTWILFLV AAATRVHSSQ CVNLTTRTQL PPAYTNSFTR GVYYPDKVFR SSVLHSTQDL |
| 61 | FLPFFSNVTW FHAIHVSGTN GTKRFDNPVL PFNDGVYFAS TEKSNIIRGW IFGTTLDSKT |
| 121 | QSLLIVNNAT NVVIKVCEFQ FCNDPFLGVY YHKNNKSWME SEFRVYSSAN NCTFEYVSQP |
| 181 | FLMDLEGKQG NFKNLREFVF KNIDGYFKIY SKHTPINLVR DLPQGFSALE PLVDLPIGIN |
| 241 | ITRFQTLLAL HRSYLTPGDS SSGWTAGAAA YYVGYLQPRT FLLKYNENGT ITDAVDCALD |
| 301 | PLSETKCTLK SFTVEKGIYQ TSNFRVQPTE SIVRFPNITN LCPFGEVFNA TRFASVYAWN |
| 361 | RKRISNCVAD YSVLYNSASF STFKCYGVSP TKLNDLCFTN VYADSFVIRG DEVRQIAPGQ |
| 421 | TGKIADYNYK LPDDFTGCVI AWNSNNLDSK VGGNYNYLYR LFRKSNLKPF ERDISTEIYQ |
| 481 | AGSTPCNGVE GFNCYFPLQS YGFQPTNGVG YQPYRVVVLS FELLHAPATV CGPKKSTNLV |
| 541 | KNKCVNFNFN GLTGTGVLTE SNKKFLPFQQ FGRDIADTTD AVRDPQTLEI LDITPCSFGG |
| 601 | VSVITPGTNT SNQVAVLYQD VNCTEVPVAI HADQLTPTWR VYSTGSNVFQ TRAGCLIGAE |
| 661 | HVNNSYECDI PIGAGICASY QTQTNSPRRA RSVASQSIIA YTMSLGAENS VAYSNNSIAI |
| 721 | PTNFTISVTT EILPVSMTKT SVDCTMYICG DSTECSNLLL QYGSFCTQLN RALTGIAVEQ |
| 781 | DKNTQEVFAQ VKQIYKTPPI KDFGGFNFSQ ILPDPSKPSK RSFIEDLLFN KVTLADAGFI |
| 841 | KQYGDCLGDI AARDLICAQK FNGLTVLPPL LTDEMIAQYT SALLAGTITS GWTFGAGAAL |
| 901 | QIPFAMQMAY RFNGIGVTQN VLYENQKLIA NQFNSAIGKI QDSLSSTASA LGKLQDVVNQ |
| 961 | NAQALNTLVK QLSSNFGAIS SVLNDILSRL DKVEAEVQID RLITGRLQSL QTYVTQQLIR |
| 1021 | AAEIRASANL AATKMSECVL GQSKRVDFCG KGYHLMSFPQ SAPHGVVFLH VTYVPAQEKN |
| 1081 | FTTAPAICHD GKAHFPREGV FVSNGTHWFV TQRNFYEPQI ITTDNTFVSG NCDVVIGIVN |
| 1141 | NTVYDPLQPE LDSFKEELDK YFKNHTSPDV DLGDISGINA SVVNIQKEID RLNEVAKNLN |
| 1201 | ESLIDLQELG KYEQYIKWPW YIWLGFIAGL IAIVMVTIML CCMTSCCSCL KGCCSCGSCC |
| 1261 | KFDEDDSEPV LKGVKLHYT |
| DNA insert sequence of pGX9501 (SEQ ID NO: 2) (IgE | |
| leader sequence underlined): | |
| 1 | ATGGATTGGA CTTGGATTCT CTTTCTCGTT GCTGCAGCCA CACGCGTTCA TAGCAGCCAG |
| 61 | TGTGTGAACC TGACCACCAG AACACAGCTG CCTCCTGCCT ACACCAACAG CTTCACCAGA |
| 121 | GGAGTCTACT ACCCAGACAA AGTCTTCAGA AGCTCTGTGC TGCACAGCAC CCAGGACCTG |
| 181 | TTCCTGCCTT TCTTCAGCAA CGTGACCTGG TTCCACGCCA TCCACGTGTC TGGCACCAAC |
| 241 | GGCACCAAGA GATTTGACAA CCCTGTTCTT CCTTTCAATG ATGGCGTGTA CTTTGCCAGC |
| 301 | ACAGAGAAGA GCAACATCAT CCGAGGCTGG ATCTTTGGCA CCACCCTGGA CAGCAAAACC |
| 361 | CAGAGCCTGC TGATCGTGAA CAACGCCACC AACGTGGTCA TCAAGGTGTG TGAGTTCCAG |
| 421 | TTCTGCAATG ACCCTTTCCT GGGCGTGTAC TACCACAAGA ACAACAAGTC CTGGATGGAG |
| 481 | TCTGAGTTCA GAGTCTACAG CTCTGCCAAC AACTGCACAT TTGAATATGT GTCCCAGCCT |
| 541 | TTCCTGATGG ACCTGGAGGG CAAGCAGGGC AACTTTAAGA ACCTGAGAGA ATTTGTGTTC |
| 601 | AAGAACATCG ATGGCTACTT CAAGATCTAC AGCAAGCACA CACCCATCAA CCTGGTGAGA |
| 661 | GACCTGCCTC AGGGCTTCTC TGCCCTGGAG CCTCTGGTGG ACCTGCCCAT CGGCATCAAC |
| 721 | ATCACCAGAT TCCAGACCCT GCTGGCCCTG CACAGAAGCT ACCTGACCCC AGGAGACAGC |
| 781 | AGCAGCGGCT GGACAGCTGG AGCTGCTGCC TACTACGTGG GCTACCTGCA GCCCAGGACC |
| 841 | TTCCTGCTGA AGTACAACGA AAATGGCACC ATCACAGATG CTGTTGACTG TGCCCTGGAC |
| 901 | CCTCTTAGCG AGACCAAGTG CACCCTGAAG TCCTTCACAG TGGAGAAAGG CATCTACCAG |
| 961 | ACCAGCAACT TCCGAGTGCA GCCAACAGAG AGCATCGTGA GATTTCCAAA CATCACCAAC |
| 1021 | CTGTGCCCTT TTGGAGAAGT CTTCAATGCC ACCAGATTTG CTTCTGTGTA CGCCTGGAAC |
| 1081 | AGAAAAAGAA TCAGCAACTG TGTGGCTGAC TACTCTGTGC TGTACAACTC TGCCTCCTTC |
| 1141 | TCCACCTTCA AGTGCTATGG AGTCTCTCCA ACCAAGCTGA ATGACCTGTG CTTCACCAAC |
| 1201 | GTGTATGCTG ACAGCTTTGT GATCAGAGGA GATGAAGTGC GGCAGATTGC TCCTGGCCAG |
| 1261 | ACAGGCAAGA TTGCTGACTA CAACTACAAG CTGCCTGATG ACTTCACAGG CTGTGTCATC |
| 1321 | GCCTGGAACA GCAACAACCT GGACAGCAAG GTGGGCGGCA ACTACAACTA CCTGTACAGA |
| 1381 | CTTTTCAGGA AGAGCAACCT GAAGCCTTTT GAAAGAGACA TCTCCACAGA GATCTACCAG |
| 1441 | GCTGGCAGCA CACCCTGCAA TGGTGTGGAA GGCTTCAACT GCTACTTCCC TCTGCAGAGC |
| 1501 | TACGGCTTCC AGCCAACAAA TGGCGTGGGC TACCAGCCTT ACAGAGTGGT GGTGCTGTCC |
| 1561 | TTTGAGCTGC TGCACGCCCC TGCCACAGTG TGTGGCCCCA AGAAGAGCAC CAACCTGGTG |
| 1621 | AAGAACAAAT GTGTGAACTT CAATTTCAAT GGCCTGACAG GCACAGGAGT GCTGACAGAG |
| 1681 | AGCAACAAGA AGTTTCTTCC TTTCCAGCAG TTTGGAAGAG ACATTGCTGA CACCACAGAT |
| 1741 | GCTGTGAGAG ATCCTCAGAC CCTGGAGATC CTGGATATCA CACCCTGCTC CTTTGGAGGA |
| 1801 | GTTTCTGTCA TCACACCTGG CACCAATACC AGCAACCAAG TGGCTGTGCT GTACCAAGAT |
| 1861 | GTGAATTGCA CAGAAGTGCC TGTGGCCATC CACGCTGACC AGCTGACACC CACCTGGAGA |
| 1921 | GTGTACAGCA CAGGCAGCAA TGTTTTCCAG ACAAGAGCTG GCTGCCTGAT TGGAGCAGAG |
| 1981 | CACGTGAACA ACAGCTATGA ATGTGACATC CCTATTGGAG CTGGCATCTG TGCCAGCTAC |
| 2041 | CAGACCCAAA CCAACAGCCC AAGAAGAGCC AGATCTGTGG CCAGCCAGAG CATCATCGCC |
| 2101 | TACACCATGA GCCTGGGAGC TGAGAACTCT GTGGCCTACA GCAACAACAG CATCGCCATC |
| 2161 | CCCACCAACT TCACCATCTC TGTGACCACA GAGATCCTGC CTGTGTCCAT GACCAAGACA |
| 2221 | TCTGTGGACT GCACCATGTA CATCTGTGGA GACAGCACAG AATGCAGCAA CCTGCTGCTG |
| 2281 | CAGTACGGCT CCTTCTGCAC CCAGCTGAAC AGAGCCCTGA CAGGCATCGC TGTGGAGCAG |
| 2341 | GACAAGAACA CACAGGAAGT GTTTGCCCAG GTGAAGCAGA TCTACAAAAC ACCACCCATC |
| 2401 | AAGGACTTTG GAGGCTTCAA TTTCTCCCAA ATCCTGCCTG ACCCCAGCAA GCCTTCCAAG |
| 2461 | AGAAGCTTCA TTGAAGACCT GCTGTTCAAC AAAGTGACCC TGGCTGATGC TGGCTTCATC |
| 2521 | AAGCAGTATG GAGACTGCCT GGGAGACATT GCTGCCAGAG ACCTGATCTG TGCCCAGAAG |
| 2581 | TTTAATGGCC TGACTGTGCT GCCTCCTCTG CTGACAGATG AAATGATCGC CCAGTACACA |
| 2641 | TCTGCCCTGC TGGCTGGCAC CATCACCAGT GGCTGGACAT TTGGAGCTGG AGCTGCCCTG |
| 2701 | CAGATCCCTT TTGCCATGCA GATGGCCTAC AGATTTAATG GCATCGGCGT GACCCAGAAC |
| 2761 | GTGCTGTACG AGAACCAGAA GCTGATCGCC AACCAGTTCA ACTCTGCCAT CGGCAAGATC |
| 2821 | CAGGACAGCC TGAGCAGCAC AGCCTCTGCC CTGGGCAAGC TGCAGGATGT GGTGAACCAA |
| 2881 | AACGCCCAGG CCCTGAACAC CCTGGTGAAG CAGCTGAGCA GCAACTTTGG AGCCATCTCC |
| 2941 | TCTGTGCTGA ATGACATCCT GAGCCGGCTG GACAAGGTGG AAGCAGAAGT GCAGATCGAC |
| 3001 | AGACTCATCA CAGGCCGCCT GCAGAGCCTG CAGACCTACG TGACCCAGCA GCTGATCAGA |
| 3061 | GCTGCTGAGA TCCGGGCCTC TGCCAACCTG GCTGCCACCA AGATGTCAGA ATGTGTGCTG |
| 3121 | GGCCAGAGCA AAAGAGTGGA CTTCTGTGGC AAAGGCTACC ACCTGATGTC CTTCCCTCAG |
| 3181 | TCTGCTCCTC ACGGCGTGGT GTTCCTGCAC GTGACCTACG TGCCTGCCCA GGAGAAGAAC |
| 3241 | TTCACCACAG CTCCTGCCAT CTGCCACGAT GGCAAGGCCC ACTTCCCAAG AGAAGGTGTC |
| 3301 | TTTGTGTCCA ATGGCACCCA CTGGTTCGTG ACCCAGAGAA ACTTCTACGA GCCTCAGATC |
| 3361 | ATCACCACAG ACAACACATT TGTGTCTGGC AACTGTGATG TGGTCATCGG CATCGTGAAC |
| 3421 | AACACAGTTT ATGACCCTCT GCAGCCTGAG CTGGACAGCT TCAAAGAAGA GCTGGACAAG |
| 3481 | TACTTCAAGA ACCACACATC TCCAGATGTG GACCTGGGAG ACATCTCTGG CATCAATGCC |
| 3541 | TCTGTGGTGA ACATCCAGAA GGAAATTGAC AGGCTGAACG AAGTGGCCAA GAACCTGAAC |
| 3601 | GAAAGCCTCA TCGACCTGCA GGAGCTGGGC AAGTACGAGC AGTACATCAA GTGGCCTTGG |
| 3661 | TACATCTGGC TGGGCTTCAT CGCTGGCCTC ATCGCCATCG TGATGGTGAC CATCATGCTG |
| 3721 | TGCTGCATGA CCAGCTGCTG CTCTTGCCTG AAGGGCTGCT GCAGCTGTGG CAGCTGCTGC |
| 3781 | AAGTTTGATG AAGATGACTC TGAGCCTGTG CTGAAGGGCG TGAAGCTGCA CTACACA |
| Single strand DNA sequence of pGX9501 (SEQ ID NO: 3): | |
| 1 | gctgcttcgc gatgtacggg ccagatatac gcgttgacat tgattattga ctagttatta |
| 61 | atagtaatca attacggggt cattagttca tagcccatat atggagttcc gcgttacata |
| 121 | acttacggta aatggcccgc ctggctgacc gcccaacgac ccccgcccat tgacgtcaat |
| 181 | aatgacgtat gttcccatag taacgccaat agggactttc cattgacgtc aatgggtgga |
| 241 | gtatttacgg taaactgccc acttggcagt acatcaagtg tatcatatgc caagtacgcc |
| 301 | ccctattgac gtcaatgacg gtaaatggcc cgcctggcat tatgcccagt acatgacctt |
| 361 | atgggacttt cctacttggc agtacatcta cgtattagtc atcgctatta ccatggtgat |
| 421 | gcggttttgg cagtacatca atgggcgtgg atagcggttt gactcacggg gatttccaag |
| 481 | tctccacccc attgacgtca atgggagttt gttttggcac caaaatcaac gggactttcc |
| 541 | aaaatgtcgt aacaactccg ccccattgac gcaaatgggc ggtaggcgtg tacggtggga |
| 601 | ggtctatata agcagagctc tctggctaac tagagaaccc actgcttact ggcttatcga |
| 661 | aattaatacg actcactata gggagaccca agctggctag cgtttaaact taagcttggt |
| 721 | accgagctcg gatccgccac catggattgg acttggattc tctttctcgt tgctgcagcc |
| 781 | acacgcgttc atagcagcca gtgtgtgaac ctgaccacca gaacacagct gcctcctgcc |
| 841 | tacaccaaca gcttcaccag aggagtctac tacccagaca aagtcttcag aagctctgtg |
| 901 | ctgcacagca cccaggacct gttcctgcct ttcttcagca acgtgacctg gttccacgcc |
| 961 | atccacgtgt ctggcaccaa cggcaccaag agatttgaca accctgttct tcctttcaat |
| 1021 | gatggcgtgt actttgccag cacagagaag agcaacatca tccgaggctg gatctttggc |
| 1081 | accaccctgg acagcaaaac ccagagcctg ctgatcgtga acaacgccac caacgtggtc |
| 1141 | atcaaggtgt gtgagttcca gttctgcaat gaccctttcc tgggcgtgta ctaccacaag |
| 1201 | aacaacaagt cctggatgga gtctgagttc agagtctaca gctctgccaa caactgcaca |
| 1261 | tttgaatatg tgtcccagcc tttcctgatg gacctggagg gcaagcaggg caactttaag |
| 1321 | aacctgagag aatttgtgtt caagaacatc gatggctact tcaagatcta cagcaagcac |
| 1381 | acacccatca acctggtgag agacctgcct cagggcttct ctgccctgga gcctctggtg |
| 1441 | gacctgccca tcggcatcaa catcaccaga ttccagaccc tgctggccct gcacagaagc |
| 1501 | tacctgaccc caggagacag cagcagcggc tggacagctg gagctgctgc ctactacgtg |
| 1561 | ggctacctgc agcccaggac cttcctgctg aagtacaacg aaaatggcac catcacagat |
| 1621 | gctgttgact gtgccctgga ccctcttagc gagaccaagt gcaccctgaa gtccttcaca |
| 1681 | gtggagaaag gcatctacca gaccagcaac ttccgagtgc agccaacaga gagcatcgtg |
| 1741 | agatttccaa acatcaccaa cctgtgccct tttggagaag tcttcaatgc caccagattt |
| 1801 | gcttctgtgt acgcctggaa cagaaaaaga atcagcaact gtgtggctga ctactctgtg |
| 1861 | ctgtacaact ctgcctcctt ctccaccttc aagtgctatg gagtctctcc aaccaagctg |
| 1921 | aatgacctgt gcttcaccaa cgtgtatgct gacagctttg tgatcagagg agatgaagtg |
| 1981 | cggcagattg ctcctggcca gacaggcaag attgctgact acaactacaa gctgcctgat |
| 2041 | gacttcacag gctgtgtcat cgcctggaac agcaacaacc tggacagcaa ggtgggcggc |
| 2101 | aactacaact acctgtacag acttttcagg aagagcaacc tgaagccttt tgaaagagac |
| 2161 | atctccacag agatctacca ggctggcagc acaccctgca atggtgtgga aggcttcaac |
| 2221 | tgctacttcc ctctgcagag ctacggcttc cagccaacaa atggcgtggg ctaccagcct |
| 2281 | tacagagtgg tggtgctgtc ctttgagctg ctgcacgccc ctgccacagt gtgtggcccc |
| 2341 | aagaagagca ccaacctggt gaagaacaaa tgtgtgaact tcaatttcaa tggcctgaca |
| 2401 | ggcacaggag tgctgacaga gagcaacaag aagtttcttc ctttccagca gtttggaaga |
| 2461 | gacattgctg acaccacaga tgctgtgaga gatcctcaga ccctggagat cctggatatc |
| 2521 | acaccctgct cctttggagg agtttctgtc atcacacctg gcaccaatac cagcaaccaa |
| 2581 | gtggctgtgc tgtaccaaga tgtgaattgc acagaagtgc ctgtggccat ccacgctgac |
| 2641 | cagctgacac ccacctggag agtgtacagc acaggcagca atgttttcca gacaagagct |
| 2701 | ggctgcctga ttggagcaga gcacgtgaac aacagctatg aatgtgacat ccctattgga |
| 2761 | gctggcatct gtgccagcta ccagacccaa accaacagcc caagaagagc cagatctgtg |
| 2821 | gccagccaga gcatcatcgc ctacaccatg agcctgggag ctgagaactc tgtggcctac |
| 2881 | agcaacaaca gcatcgccat ccccaccaac ttcaccatct ctgtgaccac agagatcctg |
| 2941 | cctgtgtcca tgaccaagac atctgtggac tgcaccatgt acatctgtgg agacagcaca |
| 3001 | gaatgcagca acctgctgct gcagtacggc tccttctgca cccagctgaa cagagccctg |
| 3061 | acaggcatcg ctgtggagca ggacaagaac acacaggaag tgtttgccca ggtgaagcag |
| 3121 | atctacaaaa caccacccat caaggacttt ggaggcttca atttctccca aatcctgcct |
| 3181 | gaccccagca agccttccaa gagaagcttc attgaagacc tgctgttcaa caaagtgacc |
| 3241 | ctggctgatg ctggcttcat caagcagtat ggagactgcc tgggagacat tgctgccaga |
| 3301 | gacctgatct gtgcccagaa gtttaatggc ctgactgtgc tgcctcctct gctgacagat |
| 3361 | gaaatgatcg cccagtacac atctgccctg ctggctggca ccatcaccag tggctggaca |
| 3421 | tttggagctg gagctgccct gcagatccct tttgccatgc agatggccta cagatttaat |
| 3481 | ggcatcggcg tgacccagaa cgtgctgtac gagaaccaga agctgatcgc caaccagttc |
| 3541 | aactctgcca tcggcaagat ccaggacagc ctgagcagca cagcctctgc cctgggcaag |
| 3601 | ctgcaggatg tggtgaacca aaacgcccag gccctgaaca ccctggtgaa gcagctgagc |
| 3661 | agcaactttg gagccatctc ctctgtgctg aatgacatcc tgagccggct ggacaaggtg |
| 3721 | gaagcagaag tgcagatcga cagactcatc acaggccgcc tgcagagcct gcagacctac |
| 3781 | gtgacccagc agctgatcag agctgctgag atccgggcct ctgccaacct ggctgccacc |
| 3841 | aagatgtcag aatgtgtgct gggccagagc aaaagagtgg acttctgtgg caaaggctac |
| 3901 | cacctgatgt ccttccctca gtctgctcct cacggcgtgg tgttcctgca cgtgacctac |
| 3961 | gtgcctgccc aggagaagaa cttcaccaca gctcctgcca tctgccacga tggcaaggcc |
| 4021 | cacttcccaa gagaaggtgt ctttgtgtcc aatggcaccc actggttcgt gacccagaga |
| 4081 | aacttctacg agcctcagat catcaccaca gacaacacat ttgtgtctgg caactgtgat |
| 4141 | gtggtcatcg gcatcgtgaa caacacagtt tatgaccctc tgcagcctga gctggacagc |
| 4201 | ttcaaagaag agctggacaa gtacttcaag aaccacacat ctccagatgt ggacctggga |
| 4261 | gacatctctg gcatcaatgc ctctgtggtg aacatccaga aggaaattga caggctgaac |
| 4321 | gaagtggcca agaacctgaa cgaaagcctc atcgacctgc aggagctggg caagtacgag |
| 4381 | cagtacatca agtggccttg gtacatctgg ctgggcttca tcgctggcct catcgccatc |
| 4441 | gtgatggtga ccatcatgct gtgctgcatg accagctgct gctcttgcct gaagggctgc |
| 4501 | tgcagctgtg gcagctgctg caagtttgat gaagatgact ctgagcctgt gctgaagggc |
| 4561 | gtgaagctgc actacacatg ataactcgag tctagagggc ccgtttaaac ccgctgatca |
| 4621 | gcctcgactg tgccttctag ttgccagcca tctgttgttt gcccctcccc cgtgccttcc |
| 4681 | ttgaccctgg aaggtgccac tcccactgtc ctttcctaat aaaatgagga aattgcatcg |
| 4741 | cattgtctga gtaggtgtca ttctattctg gggggtgggg tggggcagga cagcaagggg |
| 4801 | gaggattggg aagacaatag caggcatgct ggggatgcgg tgggctctat ggcttctact |
| 4861 | gggcggtttt atggacagca agcgaaccgg aattgccagc tggggcgccc tctggtaagg |
| 4921 | ttgggaagcc ctgcaaagta aactggatgg ctttcttgcc gccaaggatc tgatggcgca |
| 4981 | ggggatcaag ctctgatcaa gagacaggat gaggatcgtt tcgcatgatt gaacaagatg |
| 5041 | gattgcacgc aggttctccg gccgcttggg tggagaggct attcggctat gactgggcac |
| 5101 | aacagacaat cggctgctct gatgccgccg tgttccggct gtcagcgcag gggcgcccgg |
| 5161 | ttctttttgt caagaccgac ctgtccggtg ccctgaatga actgcaagac gaggcagcgc |
| 5221 | ggctatcgtg gctggccacg acgggcgttc cttgcgcagc tgtgctcgac gttgtcactg |
| 5281 | aagcgggaag ggactggctg ctattgggcg aagtgccggg gcaggatctc ctgtcatctc |
| 5341 | accttgctcc tgccgagaaa gtatccatca tggctgatgc aatgcggcgg ctgcatacgc |
| 5401 | ttgatccggc tacctgccca ttcgaccacc aagcgaaaca tcgcatcgag cgagcacgta |
| 5461 | ctcggatgga agccggtctt gtcgatcagg atgatctgga cgaagagcat caggggctcg |
| 5521 | cgccagccga actgttcgcc aggctcaagg cgagcatgcc cgacggcgag gatctcgtcg |
| 5581 | tgacccatgg cgatgcctgc ttgccgaata tcatggtgga aaatggccgc ttttctggat |
| 5641 | tcatcgactg tggccggctg ggtgtggcgg accgctatca ggacatagcg ttggctaccc |
| 5701 | gtgatattgc tgaagagctt ggcggcgaat gggctgaccg cttcctcgtg ctttacggta |
| 5761 | tcgccgctcc cgattcgcag cgcatcgcct tctatcgcct tcttgacgag ttcttctgaa |
| 5821 | ttattaacgc ttacaatttc ctgatgcggt attttctcct tacgcatctg tgcggtattt |
| 5881 | cacaccgcat caggtggcac ttttcgggga aatgtgcgcg gaacccctat ttgtttattt |
| 5941 | ttctaaatac attcaaatat gtatccgctc atgagacaat aaccctgata aatgcttcaa |
| 6001 | taatagcacg tgctaaaact tcatttttaa tttaaaagga tctaggtgaa gatccttttt |
| 6061 | gataatctca tgaccaaaat cccttaacgt gagttttcgt tccactgagc gtcagacccc |
| 6121 | gtagaaaaga tcaaaggatc ttcttgagat cctttttttc tgcgcgtaat ctgctgcttg |
| 6181 | caaacaaaaa aaccaccgct accagcggtg gtttgtttgc cggatcaaga gctaccaact |
| 6241 | ctttttccga aggtaactgg cttcagcaga gcgcagatac caaatactgt tcttctagtg |
| 6301 | tagccgtagt taggccacca cttcaagaac tctgtagcac cgcctacata cctcgctctg |
| 6361 | ctaatcctgt taccagtggc tgctgccagt ggcgataagt cgtgtcttac cgggttggac |
| 6421 | tcaagacgat agttaccgga taaggcgcag cggtcgggct gaacgggggg ttcgtgcaca |
| 6481 | cagcccagct tggagcgaac gacctacacc gaactgagat acctacagcg tgagctatga |
| 6541 | gaaagcgcca cgcttcccga agggagaaag gcggacaggt atccggtaag cggcagggtc |
| 6601 | ggaacaggag agcgcacgag ggagcttcca gggggaaacg cctggtatct ttatagtcct |
| 6661 | gtcgggtttc gccacctctg acttgagcgt cgatttttgt gatgctcgtc aggggggcgg |
| 6721 | agcctatgga aaaacgccag caacgcggcc tttttacggt tcctggcctt ttgctggcct |
| 6781 | tttgctcaca tgttctt |

What is claimed is:

1. A method of inducing an immune response against Severe Acute Respiratory Syndrome coronavirus 2 (SARS-CoV-2) variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 in a subject in need thereof, the method comprising administering to the subject an effective amount of pGX9501, INO-4800 drug product, or a biosimilar thereof.

2. The method of claim 1, wherein the immune response is a humoral immune response, a cellular immune response, or both.

3. The method of claim 1, wherein the subject is thereby protected against infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529.

4. The method of claim 1, wherein the subject is thereby treated for a disease or disorder associated with infection by SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529.

5. The method of claim 4, wherein the disease or disorder associated with infection by (SARS-CoV-2) variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529 is Coronavirus Disease 2019 (COVID-19), Multisystem inflammatory syndrome in adults (MIS-A), or Multisystem inflammatory syndrome in children (MIS-C).

6. The method of claim 1, wherein administering comprises at least one of electroporation and parenteral administration.

7. The method of claim 6, wherein administering comprises parenteral administration followed by electroporation.

8. The method of claim 6 wherein the parenteral administration is subcutaneous administration, intradermal administration, or intramuscular administration.

9. The method of claim 1, wherein an initial dose of about 0.5 mg to about 2.0 mg of pGX9501 or nucleic acid component of INO-4800 or a biosimilar thereof is administered to the subject.

10. The method of claim 1, wherein an initial dose of about 0.5 mg, about 1.0 mg or about 2.0 mg of pGX9501 or nucleic acid component of INO-4800 or a biosimilar thereof is administered to the subject.

11. The method of claim 9, wherein a subsequent dose of about 0.5 mg to about 2.0 mg of pGX9501 or nucleic acid component of INO-4800 or a biosimilar thereof is administered to the subject about four weeks after the initial dose.

12. The method of claim 9, wherein a subsequent dose of about 0.5 mg, about 1.0 mg or about 2.0 mg of pGX9501 or nucleic acid component of INO-4800 or a biosimilar thereof is administered to the subject about four weeks after the initial dose.

13. The method of claim 11, wherein one or more further subsequent doses of about 0.5 mg to about 2.0 mg of pGX9501 or nucleic acid component of INO-4800 or a biosimilar thereof is administered to the subject at least twelve weeks after the initial dose.

14. The method of claim 11, wherein one or more further subsequent doses of about 0.5 mg, about 1.0 mg, or about 2.0 mg of pGX9501 or nucleic acid component of INO-4800 or a biosimilar thereof is administered to the subject at least twelve weeks after the initial dose.

15. The method of claim 1, further comprising administering to the subject at least one additional agent for the treatment of SARS-CoV-2 infection or the treatment or prevention of a disease or disorder associated with SARS-CoV-2 infection.

16. The method of claim 15, wherein the pGX9501, INO-4800 drug product, or biosimilar thereof is administered to the subject before, concurrently with, or after the additional agent.

17. A method of protecting a subject in need thereof from infection with Severe Acute Respiratory Syndrome coronavirus 2 (SARS-CoV-2) variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529, the method comprising administering to the subject an effective amount of pGX9501, INO-4800 drug product, or a biosimilar thereof, wherein the subject is thereby resistant to one or more of SARS-CoV-2 variant B.1.351, SARS-CoV-2 variant B.1.1.7, SARS-CoV-2 variant P.1, SARS-CoV-2 variant B.1.617.1, SARS-CoV-2 variant B.1.617.2, or SARS-CoV-2 variant B.1.1.529.

18. The method of claim 17, wherein administering comprises at least one of electroporation and parenteral administration.

19. The method of claim 18, wherein administering comprises parenteral administration followed by electroporation.

20. The method of claim 18 wherein the parenteral administration is subcutaneous administration, intradermal administration, or intramuscular administration.

21. The method of claim 17, wherein an initial dose of about 0.5 mg to about 2.0 mg of pGX9501 or nucleic acid component of INO-4800 or a biosimilar thereof is administered to the subject.

22. The method of claim 17, wherein an initial dose of about 0.5 mg, about 1.0 mg or about 2.0 mg of pGX9501 or nucleic acid component of INO-4800 or a biosimilar thereof is administered to the subject.

23. The method of claim 21, wherein a subsequent dose of about 0.5 mg to about 2.0 mg of pGX9501 or nucleic acid component of INO-4800 or a biosimilar thereof is administered to the subject about four weeks after the initial dose.

24. The method of claim 21, wherein a subsequent dose of about 0.5 mg, about 1.0 mg or about 2.0 mg of pGX9501 or nucleic acid component of INO-4800 or a biosimilar thereof is administered to the subject about four weeks after the initial dose.

25. The method of claim 23, wherein one or more further subsequent doses of about 0.5 mg to about 2.0 mg of pGX9501 or nucleic acid component of INO-4800 or a biosimilar thereof is administered to the subject at least twelve weeks after the initial dose.

26. The method of claim 23, wherein one or more further subsequent doses of about 0.5 mg, about 1.0 mg, or about 2.0 mg of pGX9501 or nucleic acid component of INO-4800 or a biosimilar thereof is administered to the subject at least twelve weeks after the initial dose.

27. The method of claim 17, further comprising administering to the subject at least one additional agent for the treatment of SARS-CoV-2 infection or the treatment or prevention of a disease or disorder associated with SARS-CoV-2 infection.

28. The method of claim 27, wherein the pGX9501, INO-4800 drug product, or biosimilar thereof is administered to the subject before, concurrently with, or after the additional agent.
